SEQUENCE LISTING (60 sequences)

SEQ ID NO: 1 - DNA, 33 bp, synthetic construct
  misc_feature 1..33: note = Reverse primer of adaptors
  source 1..33: mol_type = other DNA
gatcataagcttcctctgtgttcagtgctgatc

SEQ ID NO: 2 - DNA, 96 bp, synthetic construct
  misc_feature 1..96: note = Forwardprimer R1-Adaptor-HindIII
  source 1..96: mol_type = other DNA
gcttccgtcggatttgcaagactcgcgaggatacgtgtacgaaaccagaagacgtatgcactaggtcaacaatgaagcggccgttctagttgccag

SEQ ID NO: 3 - DNA, 95 bp, synthetic construct
  misc_feature 1..95: note = Forwardprimer R7-Adaptor-HindIII
  source 1..95: mol_type = other DNA
gcttcctaacagcgccggtgggagggtaatcagaagactctatcgcgacgctatcgggtcgtattataggatgaagcggccgttctagttgccag

SEQ ID NO: 4 - DNA, 98 bp, synthetic construct
  misc_feature 1..98: note = Forwardprimer R9-Adaptor-HindIII
  source 1..98: mol_type = other DNA
tcgccaggatgccggtcagcatatatcgatactcaaggcaggtcaattcgcactgtgagggtcacatgggcgtttggcacggccgttctagttgccag

SEQ ID NO: 5 - DNA, 88 bp, synthetic construct
  misc_feature 1..88: note = Forwardprimer R11-Adaptor-HindIII
  source 1..88: mol_type = other DNA
accgacctgggttcggcactgtgggcagtgtgaggtattggcagacgcccagtgccgaacaacacctgacggccgttctagttgccag

SEQ ID NO: 6 - DNA, 88 bp, synthetic construct
  misc_feature 1..88: note = Forwardprimer R15-Adaptor-HindIII
  source 1..88: mol_type = other DNA
gctacttgatgtcttgcgacgttcttagagatggacgaaatgtttcgcgacccaggatgaggtcgccctaggccgttctagttgccag

SEQ ID NO: 7 - DNA, 88 bp, synthetic construct
  misc_feature 1..88: note = Forwardprimer R19-Adaptor-HindIII
  source 1..88: mol_type = other DNA
atcggtgggagtattcaacgtgatgaagacgctgggttcacgtgggaatggtgcttctgtcctaacaggcggccgttctagttgccag

SEQ ID NO: 8 - DNA, 34 bp, synthetic construct
  misc_feature 1..34: note = Forward primer, fragment1
  source 1..34: mol_type = other DNA
gatcatggatccgttgacattgattattgactag

SEQ ID NO: 9 - DNA, 36 bp, synthetic construct
  misc_feature 1..36: note = Reverse primer, fragment1
  source 1..36: mol_type = other DNA
gatcatgggcccaagcttcctctgtgttcagtgctg

SEQ ID NO: 10 - DNA, 108 bp, synthetic construct
  misc_feature 1..108: note = Forward primer, fragment2
  source 1..108: mol_type = other DNA
gatcatgggcccgcttccgtcggatttgcaagactcgcgaggatacgtgtacgaaaccagaagacgtatgcactaggtcaacaatgaagcggccgttctagttgccag

SEQ ID NO: 11 - DNA, 35 bp, synthetic construct
  misc_feature 1..35: note = Reverse primer, fragment2, fragment3
  source 1..35: mol_type = other DNA
gatcatttaattaagactgacgcgtcctcaaaaat

SEQ ID NO: 12 - DNA, 100 bp, synthetic construct
  misc_feature 1..100: note = Forward primer, fragment3
  source 1..100: mol_type = other DNA
ctggcaactagaacggccgcctgttaggacagaagcaccattcccacgtgaacccagcgtcttcatcacgttgaatactcccaccgatgggcccatgatc

SEQ ID NO: 13 - DNA, 17 bp, synthetic construct
  misc_feature 1..17: note = Forward primer, HindIII-DNAblock-Rx
  source 1..17: mol_type = other DNA
gaggaagcttgccgcca

SEQ ID NO: 14 - DNA, 95 bp, synthetic construct
  misc_feature 1..95: note = Reverse primer, DNAblock HindIII-xLC-R15
  source 1..95: mol_type = other DNA
tagggcgacctcatcctgggtcgcgaaacatttcgtccatctctaagaacgtcgcaagacatcaagtagctattcaactaatggcttatgtacgg

SEQ ID NO: 15 - DNA, 95 bp, synthetic construct
  misc_feature 1..95: note = Reverse primer, DNAblock BsaXI-hole-R11
  source 1..95: mol_type = other DNA
gtcaggtgttgttcggcactgggcgtctgccaatacctcacactgcccacagtgccgaacccaggtcggttattcaactaatggcttatgtacgg

SEQ ID NO: 16 - DNA, 103 bp, synthetic construct
  misc_feature 1..103: note = Reverse primer, DNAblock BsaXI-xLC-R1
  source 1..103: mol_type = other DNA
gcttcattgttgacctagtgcatacgtcttctggtttcgtacacgtatcctcgcgagtcttgcaaatccgacggaagctattcaactaatggcttatgtacgg

SEQ ID NO: 17 - DNA, 105 bp, synthetic construct
  misc_feature 1..105: note = Reverse primer, DNAblock HindIII-LC-R9/ BsaXI-xLC-R9
  source 1..105: mol_type = other DNA
gtgccaaacgcccatgtgaccctcacagtgcgaattgacctgccttgagtatcgatatatgctgaccggcatcctggcgatattcaactaatggcttatgtacgg

SEQ ID NO: 18 - DNA, 102 bp, synthetic construct
  misc_feature 1..102: note = Reverse primer, DNAblock BsaXI-knob-R7
  source 1..102: mol_type = other DNA
gcttcatcctataatacgacccgatagcgtcgcgatagagtcttctgattaccctcccaccggcgctgttaggaagctattcaactaatggcttatgtacgg

SEQ ID NO: 19 - DNA, 103 bp, synthetic construct; identical to SEQ ID NO: 16
  misc_feature 1..103: note = Reverse primer, DNAblock HindIII-knob-R1
  source 1..103: mol_type = other DNA

SEQ ID NO: 20 - DNA, 95 bp, synthetic construct
  misc_feature 1..95: note = Reverse primer, DNAblock HindIII-hole-R19/ BsaXI-LC-R19
  source 1..95: mol_type = other DNA
gcctgttaggacagaagcaccattcccacgtgaacccagcgtcttcatcacgttgaatactcccaccgattattcaactaatggcttatgtacgg

SEQ ID NO: 21 - DNA, 55 bp, synthetic construct
  misc_feature 1..55: note = Reverse primer
  source 1..55: mol_type = other DNA
caacggcggatggagtaacagttcatatcttccccctctgtgttcagtgctgatc

SEQ ID NO: 22 - DNA, 97 bp, synthetic construct
  misc_feature 1..97: note = Forward primer, R7-Adaptor-BsaXI
  source 1..97: mol_type = other DNA
gcttcctaacagcgccggtgggagggtaatcagaagactctatcgcgacgctatcgggtcgtattataggatgaagcgtacgttctagttgccagcc

SEQ ID NO: 23 - DNA, 100 bp, synthetic construct
  misc_feature 1..100: note = Forward primer, R9-Adaptor-BsaXI
  source 1..100: mol_type = other DNA
tcgccaggatgccggtcagcatatatcgatactcaaggcaggtcaattcgcactgtgagggtcacatgggcgtttggcacgtacgttctagttgccagcc

SEQ ID NO: 24 - DNA, 90 bp, synthetic construct
  misc_feature 1..90: note = Forward primer, R11-Adaptor-BsaXI
  source 1..90: mol_type = other DNA
accgacctgggttcggcactgtgggcagtgtgaggtattggcagacgcccagtgccgaacaacacctgacgtacgttctagttgccagcc

SEQ ID NO: 25 - DNA, 50 bp, synthetic construct
  misc_feature 1..50: note = Forward primer, BsaXI-DNAblock-Rx
  source 1..50: mol_type = other DNA
gtctaagatatgaactgttactccgccgttaggggccgccaccatgggat

SEQ ID NO: 26-34 - skipped (9 entries)

SEQ ID NO: 35 - DNA, 13 bp, synthetic construct
  misc_feature 1..13: note = LoxP-site palindrom
  source 1..13: mol_type = other DNA
ataacttcgtata

SEQ ID NO: 36 - DNA, 13 bp, synthetic construct
  misc_feature 1..13: note = LoxP-site palindom 2
  source 1..13: mol_type = other DNA
tatacgaagttat

SEQ ID NO: 37 - DNA, 34 bp, synthetic construct
  misc_feature 1..34: note = L3
  source 1..34: mol_type = other DNA
ataacttcgtataaagtctcctatacgaagttat

SEQ ID NO: 38 - DNA, 34 bp, synthetic construct
  misc_feature 1..34: note = 2L
  source 1..34: mol_type = other DNA
ataacttcgtatagcatacattatacgaagttat

SEQ ID NO: 39 - DNA, 34 bp, synthetic construct
  misc_feature 1..34: note = loxFas
  source 1..34: mol_type = other DNA
acaacttcgtatatacctttctatacgaagttgt

SEQ ID NO: 40 - DNA, 608 bp, Human cytomegalovirus
  source 1..608: mol_type = unassigned DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatc

SEQ ID NO: 41 - DNA, 696 bp, Human cytomegalovirus
  source 1..696: mol_type = unassigned DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatctagctctgggagaggagcccagcactagaagtcggcggtgtttccattcggtgatcagcactgaacacagaggaagcttgccgccacc

SEQ ID NO: 42 - DNA, 2125 bp, Human cytomegalovirus
  source 1..2125: mol_type = unassigned DNA
ctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacggtttaaacgccgccacc

SEQ ID NO: 43 - DNA, 129 bp, Simian virus 40
  source 1..129: mol_type = unassigned DNA
aacttgtttattgcagcttataatggttacaaataaagcaatagcatcacaaatttcacaaataaagcatttttttcaccattctagttgtggtttgtccaaactcatcaatgtatcttatcatgtctg

SEQ ID NO: 44 - DNA, 225 bp, Bos taurus
  source 1..225: mol_type = unassigned DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 45 - DNA, 73 bp, Homo sapiens
  source 1..73: mol_type = unassigned DNA
caggataatatatggtagggttcatagccagagtaacctttttttttaatttttattttattttatttttgag

SEQ ID NO: 46 - DNA, 288 bp, Simian virus 40
  source 1..288: mol_type = unassigned DNA
agtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcg

SEQ ID NO: 47 - DNA, 798 bp, synthetic construct
  misc_feature 1..798: note = green fluorescent protein encoding nucleic acid
  source 1..798: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagatga

SEQ ID NO: 48 - protein, 343 aa, Bacteriophage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 49 - RNA, 1029 nt, Bacteriophage P1
  source 1..1029: mol_type = unassigned RNA
atgagcaacctgctgaccgtgcaccagaacctgcccgccctgcccgtggacgccaccagcgacgaggtgaggaagaacctgatggacatgttcagggacaggcaggccttcagcgagcacacctggaagatgctgctgagcgtgtgcaggagctgggccgcctggtgcaagctgaacaacaggaagtggttccccgccgagcccgaggacgtgagggactacctgctgtacctgcaggccaggggcctggccgtgaagaccatccagcagcacctgggccagctgaacatgctgcacaggaggagcggcctgcccaggcccagcgacagcaacgccgtgagcctggtgatgaggaggatcaggaaggagaacgtggacgccggcgagagggccaagcaggccctggccttcgagaggaccgacttcgaccaggtgaggagcctgatggagaacagcgacaggtgccaggacatcaggaacctggccttcctgggcatcgcctacaacaccctgctgaggatcgccgagatcgccaggatcagggtgaaggacatcagcaggaccgacggcggcaggatgctgatccacatcggcaggaccaagaccctggtgagcaccgccggcgtggagaaggccctgagcctgggcgtgaccaagctggtggagaggtggatcagcgtgagcggcgtggccgacgaccccaacaactacctgttctgcagggtgaggaagaacggcgtggccgcccccagcgccaccagccagctgagcaccagggccctggagggcatcttcgaggccacccacaggctgatctacggcgccaaggacgacagcggccagaggtacctggcctggagcggccacagcgccagggtgggcgccgccagggacatggccagggccggcgtgagcatccccgagatcatgcaggccggcggctggaccaacgtgaacatcgtgatgaactacatcaggaacctggacagcgagaccggcgccatggtgaggctgctggaggacggcgac

SEQ ID NO: 50 - DNA, 13 bp, Bacteriophage P1
  source 1..13: mol_type = unassigned DNA
ataacttcgtata

SEQ ID NO: 51 - DNA, 13 bp, Bacteriophage P1
  source 1..13: mol_type = unassigned DNA
tatacgaagttat

SEQ ID NO: 52-60 - skipped (9 entries)

(19) United States
(12) Patent Application Publication
(10) Pub. No.: US 2024/0002538 A1
(43) Pub. Date: Jan. 4, 2024

(54) METHOD FOR THE ASSEMBLY OF LARGE NUCLEIC ACIDS FROM SHORT FRAGMENTS

(71) Applicant: Hoffmann-La Roche Inc., Little Falls, NJ (US)

(72) Inventors: Johannes Auer, Schwaigen (DE); Simon Auslaender, Wolfratshausen (DE); Peter Michael Huelsmann, Habach (DE)

(73) Assignee: Haffmann-la Roche Inc., Little Falls, NJ (US)

(21) Appl. No.: 17/811,550

(22) Filed: Jul. 8, 2022

Related U.S. Application Data
(63) Continuation of application No. PCT/EP2021/050241, filed on Jan. 8, 2021.

(30) Foreign Application Priority Data
Jan. 10, 2020 (EP) 20151080.7

(51) Int. Cl. C07K 16/46; C12N 15/63; C12N 9/22; C12N 15/11

(52) U.S. Cl. CPC C07K 16/468; C12N 15/63; C12N 9/22; C12N 15/11; C07K 2317/14; C07K 2317/31

(57) ABSTRACT

Herein a modular and efficient method for cloning multigene plasmids containing only a single cloning step without the use of preliminary single-gene vectors is reported. In the first step, PCR-produced DNAblocks carrying genes for different antibody chains are ligated with the respective adaptors, which serve as connectors of the different DNAblocks. In the second step, the PCR-produced fragments of the first step are assembled in the correct arrangement via the unique recombination sites located at one end of each backbone, DNAblock and adaptor fragment. This new strategy results in a modular and efficient method, which allows direct cloning of expression cassettes into the respective backbone without intermediate cloning steps and enable fast cloning of variable gene configurations of diverse antibody formats. This new cloning method according to the current invention provides considerable advantages in terms of time, work labor and costs.

### CROSS REFERENCE TO RELATED APPLICATIONS

This application is a continuation of International Application No. PCT/EP2021/050241 having an international filing date of Jan. 8, 2021, and which claims benefit of priority to European Patent Application No. 20151080.7, filed Jan. 10, 2020; all of which are incorporated herein by reference in their entirety.

### SEQUENCE LISTING

This application contains a Sequence Listing which has been submitted electronically and is hereby incorporated by reference in its entirety. It was created on July 8, is named P35845-US_Sequence_Listing.xml and is 61,525 bytes in size.

### FIELD OF THE INVENTION

Herein a modular and efficient method for cloning multigene plasmids containing only a single cloning step without the use of preliminary single-gene vectors is reported. In the first step, PCR-produced DNAblocks carrying genes for different antibody chains are ligated with the respective adaptors, which serve as connectors of the different DNA blocks. In the second step, the PCR-produced fragments of the first step are assembled in the correct arrangement via the unique recombination sites located at one end of each backbone, DNAblock and adaptor fragment. This new strategy results in a modular and efficient method, which allows direct cloning of expression cassettes into the respective backbone without intermediate cloning steps and enable fast cloning of variable gene configurations of diverse antibody formats. This new cloning method according to the current invention provides considerable advantages in terms of time, work labor and costs.

### BACKGROUND OF THE INVENTION

Therapeutic monoclonal antibodies are standard-of-care for the treatment of many types of cancer. For the manufacturing of therapeutic antibodies, CHO cells are the most common producer cell lines with the ability to express complex glycoproteins requiring posttranslational modifications. To integrate the required DNA sequences coding for the antibody components in a predetermined locus of the CHO genome, a targeted integration (TI-) system was developed.

Celie, P. H. N., et al., reported about recombinant cloning strategies for protein expression (Curr. Opin. Struct. Biol. 38 (2016) 145-154).

Betton reported about high throughput cloning and expression strategies for protein production (Biochimie 86 (2004) 601-605).

Chaudhary, V. K., et al., reported about rapid restriction enzyme-free cloning of PCR products, a high-throughput method applicable for library construction (PLOS One 9 (2014) e111538).

Tan, L., et al., reported about homologous alignment cloning, which is a rapid, flexible and highly efficient general molecular cloning method (PEER J. 6 (2018) e5146).

### SUMMARY OF THE INVENTION

When employing a TI-approach for the generation of the production cell lines, a front and a back vector are used carrying the various gene expression cassettes of the antibody chains. The cloning procedure of TI front and back vectors is generally based on the preliminary cloning of single-gene vectors coding for specific antibody chains. The corresponding gene expression cassette for the specific chains of the antibody are then cloned onto the respective multi-gene front or back vector, which are subsequently transfected into the host cell. The cloning of single-gene vectors represents a time-consuming and labor-intensive process step within projects with a high investment in resources.

In the current invention, an improved method, i.e. a time-, effort- and resource-saving cloning strategy, for creating multigene vector systems is reported. In the method according to the current invention, the use of preliminary single-gene vectors is no longer necessary. This TI-cloning method according to the invention is based on a modular system vector backbones and adaptors fragments as pre-defined generic parts and DNAblocks (antibody chain sequences) as project-specific parts. It has been found that it is advantageous to use adaptor fragments as connectors, which contain the terminator of the previous DNAblock and the promotor of the following one. Thereby the correct arrangement of the DNAblocks is ensured. In the method according to the invention, adaptors and DNAblocks are modulated by PCR to carry unique recombination sites (R-sites) at the 3′-end and a defined RE recognition site at the 5′-end of the fragment. The TI-front/back basic vectors also comprised the corresponding R-site and RE recognition sites flanking the backbones.

The invention is based, at least in part on the finding that with the generation of these generic parts (adaptors, backbones) and variable DNAblocks, whereas each of the fragment includes a specific R-site at the one end and a defined RE recognition site at the other end of the fragment, the cloning of TI-vectors is improved. Depending on the desired gene configuration, these parts are ligated and assembled to the final TI-vectors using restriction enzymes as well as the assembly of the homologous R-sites of two fragments. Due to the ability to generate most of the required sequences via PCR, this method reduces time and effort for gene synthesis as just the variable DNAblocks need to be supplied.

One aspect according to the invention is a method for producing an expression vector comprising the following step
incubating
   a linear expression vector backbone comprising
      at its 3′-end a promoter and a first single stranded enzymatic restriction site of a first restriction enzyme,
      at its 5′-end a single stranded recombination site,
   a first DNAblock comprising in 5′- to 3′-direction
      a first single stranded enzymatic restriction site of a first restriction enzyme,
      a nucleic acid encoding a protein of interest,
      a first single stranded recombination site,
   a first adaptor nucleic acid comprising in 5′- to 3′-direction
      a first single stranded recombination site,
      a polyA signal sequence,
      a promoter,
      a second single stranded enzymatic restriction site of a second restriction enzyme,
   a second DNAblock comprising in 5′- to 3′-direction
      a second single stranded enzymatic restriction site of a second restriction enzyme,
      a nucleic acid encoding a second protein of interest,
      a second single stranded recombination site, with a DNA ligase,
whereby the first enzymatic restriction site is different from the second enzymatic restriction site,
whereby the second enzymatic restriction site is of a type IIB restriction enzyme,
whereby the first and the second recombination sites are different,
whereby the second recombination site and the recombination site at the 5′-end of the vector backbone are identical,
wherein each recombination site is a 15-80 bp long nucleic acid sequence unique in the incubated nucleic acids,
wherein the first single stranded enzymatic restriction site of the linear vector backbone is capable of specifically hybridizing with the first single stranded enzymatic restriction site of the first DNAblock, the first single stranded recombination site of the first DNAblock is capable of specifically hybridizing with first single stranded recombination site of the first adaptor nucleic acid, the second single stranded enzymatic restriction site of the first adaptor nucleic acid is capable of specifically hybridizing with the second single stranded enzymatic restriction site of the second DNAblock, and the second single stranded recombination site of the second DNAblock is capable of specifically hybridizing with the single stranded recombination site of the linear vector backbone.

In one embodiment, the second enzymatic restriction site is a BsaXI restriction site.

In one embodiment, the protein of interest is an antibody chain. In one embodiment, the first protein of interest is an antibody light chain and the second protein of interest is an antibody heavy chain or vice versa.

One aspect according to the invention is a nucleic acid comprising the following elements:
an enzymatic restriction site
a first expression cassette for a first protein of interest
a first recombination site
an enzymatic restriction site for a type IIB restriction enzyme
a second expression cassette for a second protein of interest
a second recombination site,
wherein the first recombination site is different from the second recombination site,
wherein the enzymatic restriction site is different or identical to the enzymatic restriction site for a type IIB restriction enzyme.

In one embodiment, the second enzymatic restriction site is a BsaXI restriction site.

In one embodiment, the protein of interest is an antibody chain. In one embodiment, the first protein of interest is an antibody light chain and the second protein of interest is an antibody heavy chain or vice versa.

One aspect according to the invention is a cell comprising the nucleic acid according to the invention.

In one embodiment, the cell is a mammalian cell. In one embodiment, the mammalian cell is a CHO cell.

One aspect according to the invention is a method for producing an antibody comprising the following steps:
a) cultivating a mammalian cell comprising the nucleic acid according to the invention, and
b) recovering the antibody from the cell or the cultivation medium,
c) optionally purifying the antibody with one or more chromatography steps, thereby producing the antibody.

### General Description and Definitions

### Definitions

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and 11 (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N. Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In one embodiment the term about denotes a range of +/−10% of the thereafter-following numerical value. In one embodiment the term about denotes a range of +/−5% of the thereafter-following numerical value.

The term “Cre-recombinase” denotes a tyrosine recombinase that catalyzes site-specific recombinase using a topoisomerase I-like mechanism between LoxP-sites. The molecular weight of the enzyme is about 38 kDa and it consists of 343 amino acid residues. It is a member of the integrase family.

The term “comprising” also encompasses the term “consisting of”.

The term “mammalian cell comprising an exogenous nucleotide sequence” encompasses cells into which one or more exogenous nucleic acid(s) have been introduced, including the progeny of such cells and which are intended to form the starting point for further genetic modification. Thus, the term “a mammalian cell comprising an exogenous nucleotide sequence” encompasses a cell comprising an exogenous nucleotide sequence integrated at a single site within a locus of the genome of the mammalian cell, wherein the exogenous nucleotide sequence comprises at least a first and a second recombination recognition sequence (these recombinase recognition sequences are different) flanking at least one first selection marker. In one embodiment the mammalian cell comprising an exogenous nucleotide sequence is a cell comprising an exogenous nucleotide sequence integrated at a single site within a locus of the genome of the host cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different

The term “recombinant cell” as used herein denotes a cell after final genetic modification, such as, e.g., a cell expressing a polypeptide of interest and that can be used for the production of said polypeptide of interest at any scale. For example, “a mammalian cell comprising an exogenous nucleotide sequence” that has been subjected to recombinase mediated cassette exchange (RMCE) whereby the coding sequences for a polypeptide of interest have been introduced into the genome of the host cell is a “recombinant cell”. Although the cell is still capable of performing further RMCE reactions, it is not intended to do so.

The term “LoxP-site” denotes a nucleotide sequence of are 34 bp in length consisting of two palindromic 13 bp sequences at the termini (ATAACTTCGTATA (SEQ ID NO: 35) and TATACGAAGTTAT (SEQ ID NO: 36), respectively) and a central 8 bp core (not symmetric) spacer sequence. The core spacer sequences determine the orientation of the LoxP-site. Depending on the relative orientation and location of the LoxP sites with respect to each other, the intervening DNA is either excised (LoxP-sites oriented in the same direction) or inverted (LoxP-sites orientated in opposite directions). The term “floxed” denotes a DNA sequence located between two LoxP-sites. If there are two floxed sequences, i.e. a target floxed sequence in the genome and a floxed sequence in a donor nucleic acid both sequences can be exchanged with each other. This is called “recombinase-mediated cassette exchange”.

Exemplary LoxP-sites are shown in the following Table:

| Name | core | SEQ ID NO: |
|---|---|---|
| wild-Type | ATGTATGC | 52 |
| L3 | AAGTCTCC | 53 |
| 2L | GCATACAT | 54 |
| LoxFas | TACCTTTC | 55 |
| lox 511 | ATGTATAC | 56 |
| lox 5171 | ATGTGTAC | 57 |
| lox 2272 | AAGTATCC | 58 |
| M2 | AGAAACCA | 59 |
| M3 | TAATACCA | 60 |

A “mammalian cell comprising an exogenous nucleotide sequence” and a “recombinant cell” are both “transformed cells”. This term includes the primary transformed cell as well as progeny derived therefrom without regard to the number of passages. Progeny may, e.g., not be completely identical in nucleic acid content to a parent cell, but may contain mutations. Mutant progeny that has the same function or biological activity as screened or selected for in the originally transformed cell are encompassed.

An “isolated” composition is one, which has been separated from a component of its natural environment. In some embodiments, a composition is purified to greater than 95% or 99% purity as determined by, for example, electrophoretic (e.g., SDS-PAGE, isoelectric focusing (IEF), capillary electrophoresis, CE-SDS) or chromatographic (e.g., size exclusion chromatography or ion exchange or reverse phase HPLC). For review of methods for assessment of e.g. antibody purity, see, e.g., Flatman, S. et al., J. Chrom. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated” polypeptide or antibody refers to a polypeptide molecule or antibody molecule that has been separated from a component of its natural environment.

The term “integration site” denotes a nucleic acid sequence within a cell's genome into which an exogenous nucleotide sequence is inserted. In certain embodiments, an integration site is between two adjacent nucleotides in the cell's genome. In certain embodiments, an integration site includes a stretch of nucleotide sequences. In certain embodiments, the integration site is located within a specific locus of the genome of a mammalian cell. In certain embodiments, the integration site is within an endogenous gene of a mammalian cell.

The terms “vector” or “plasmid”, which can be used interchangeably, as used herein, refer to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

As used herein, the term “flanking” refers to that a first nucleotide sequence is located at either a 5′- or 3′-end, or both ends of a second nucleotide sequence. The flanking nucleotide sequence can be adjacent to or at a defined distance from the second nucleotide sequence. There is no specific limit of the length of a flanking nucleotide sequence. For example, a flanking sequence can be a few base pairs or a few thousand base pairs.

Deoxyribonucleic acids comprise a coding and a non-coding strand. The terms “5′” and “3′” when used herein refer to the position on the coding strand.

As used herein, the term “exogenous” indicates that a nucleotide sequence does not originate from a specific cell and is introduced into said cell by DNA delivery methods, e.g., by transfection, electroporation, or transformation methods. Thus, an exogenous nucleotide sequence is an artificial sequence wherein the artificiality can originate, e.g., from the combination of subsequences of different origin (e.g. a combination of a recombinase recognition sequence with an SV40 promoter and a coding sequence of green fluorescent protein is an artificial nucleic acid) or from the deletion of parts of a sequence (e.g. a sequence coding only the extracellular domain of a membrane-bound receptor or a cDNA) or the mutation of nucleobases. The term “endogenous” refers to a nucleotide sequence originating from a cell. An “exogenous” nucleotide sequence can have an “endogenous” counterpart that is identical in base compositions, but where the “exogenous” sequence is introduced into the cell, e.g., via recombinant DNA technology.

### Random Integration

The traditional method to insert plasmid DNA in the genome of a mammalian cell is the use of random plasmid integration (RI) followed by clone selection and screening. When the mammalian cell is transfected with an exogenous plasmid, integration occurs at one or a few chromosomal sites. While this approach is successfully used by industrial manufacturing CLD (cell line development), inherent disadvantages exist [1], [2]. With random integration, the expression level strongly depends on local chromosomal context and is therefore unpredictable. For example, the insertion may occur at a site in the genome that is not or weakly transcriptionally active resulting in low or no expression of the product. The high afford in screening a huge number of clones to identify high-producing clones is time and labor intensive. Moreover, productive clones may have an inconsistent and higher number of integrated transgenes. This can induce chromosomal rearrangements and may lead to repeat-induced methylation of the promotor and thus to silencing of the gene [3], [4]. Consequently, some clones become instable in presence of the selection pressure. With the increasing number of integration sites, the analysis of microheterogeniety e.g. sequence variants get more difficult. In view of all the issues, targeted integration gets a beneficial alternative to integrate transgenes in the genome of a host cell [5].

### Targeted Integration

Targeted integration (TI) results in the integration of transgenes at a single, predetermined locus within the genome of a mammalian cell [6]. Therefore, the host cell line requires or is engineered to comprise a well-known, specific recombination site placed in a locus, called “hotspot”, which guarantees high, reliable and stable production of transgenes with low or even single copy numbers [1]. Crawford et al. described in their paper how they established a stable host cell line via the combination of two technologies: the ϕC31 integrase and CRE-Lox recombinase technology. The system was used to integrate a “platform plasmid” into the CHO genome. This platform provides a base cassette containing flanking CRE-Lox recombination sites (L3 and 2L) that allow a recombinase-mediated cassette exchange (RMCE) with a plasmid encoding for antibody chains and a different resistance gene.

Thus, targeted integration allows exogenous nucleotide sequences to be integrated into a pre-determined site of a mammalian cell's genome. In certain embodiments, the targeted integration is mediated by a recombinase that recognizes one or more recombination recognition sequences (RRSs). In certain embodiments, the targeted integration is mediated by homologous recombination.

A “recombination recognition sequence” (RRS) is a nucleotide sequence recognized by a recombinase and is necessary and sufficient for recombinase-mediated recombination events. A RRS can be used to define the position where a recombination event will occur in a nucleotide sequence.

In certain embodiments, a RRS is selected from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence. If multiple RRSs have to be present, the selection of each of the sequences is dependent on the other insofar as non-identical RRSs are chosen.

In certain embodiments, a RRS can be recognized by a Cre recombinase. In certain embodiments, a RRS can be recognized by a FLP recombinase. In certain embodiments, a RRS can be recognized by a Bxb1 integrase. In certain embodiments, a RRS can be recognized by a φC31 integrase.

In certain embodiments when the RRS is a LoxP site, the cell requires the Cre recombinase to perform the recombination. In certain embodiments when the RRS is a FRT site, the cell requires the FLP recombinase to perform the recombination. In certain embodiments when the RRS is a Bxb1 attP or a Bxb1 attB site, the cell requires the Bxb1 integrase to perform the recombination. In certain embodiments when the RRS is a φC31 attP or a φC31 attB site, the cell requires the φC31 integrase to perform the recombination. The recombinases can be introduced into a cell using an expression vector comprising coding sequences of the enzymes.

The Cre-LoxP site-specific recombination system has been widely used in many biological experimental systems. Cre is a 38-kDa site-specific DNA recombinase that recognizes 34 bp LoxP sequences. Cre is derived from bacteriophage P1 and belongs to the tyrosine family site-specific recombinase. Cre recombinase can mediate both intra and intermolecular recombination between LoxP sequences. The LoxP sequence is composed of an 8 bp non-palindromic core region flanked by two 13 bp inverted repeats. Cre recombinase binds to the 13 bp repeat thereby mediating recombination within the 8 bp core region. Cre-LoxP-mediated recombination occurs at a high efficiency and does not require any other host factors. If two LoxP sequences are placed in the same orientation on the same nucleotide sequence, Cre-mediated recombination will excise DNA sequences located between the two LoxP sequences as a covalently closed circle. If two LoxP sequences are placed in an inverted position on the same nucleotide sequence, Cre-mediated recombination will invert the orientation of the DNA sequences located between the two sequences. If two LoxP sequences are on two different DNA molecules and if one DNA molecule is circular, Cre-mediated recombination will result in integration of the circular DNA sequence.

In certain embodiments, a LoxP sequence is a wild-type LoxP sequence. In certain embodiments, a LoxP sequence is a mutant LoxP sequence. Mutant LoxP sequences have been developed to increase the efficiency of Cre-mediated integration or replacement. In certain embodiments, a mutant LoxP sequence is selected from the group consisting of a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, and a Lox66 sequence. For example, the Lox71 sequence has 5 bp mutated in the left 13 bp repeat. The Lox66 sequence has 5 bp mutated in the right 13 bp repeat. Both the wild-type and the mutant LoxP sequences can mediate Cre-dependent recombination.

The term “matching RRSs” indicates that a recombination occurs between two RRSs. In certain embodiments, the two matching RRSs are the same. In certain embodiments, both RRSs are wild-type LoxP sequences. In certain embodiments, both RRSs are mutant LoxP sequences. In certain embodiments, both RRSs are wild-type FRT sequences. In certain embodiments, both RRSs are mutant FRT sequences. In certain embodiments, the two matching RRSs are different sequences but can be recognized by the same recombinase. In certain embodiments, the first matching RRS is a Bxb1 attP sequence and the second matching RRS is a Bxb1 attB sequence. In certain embodiments, the first matching RRS is a φC31 attB sequence and the second matching RRS is a φC31 attB sequence.

### Established Expression Vector Cloning Methods

Expression vectors, which are subsequently transfected into a mammalian cell, can be cloned using different methods. In general, in case of an antibody the complete gene expression cassettes for the individual antibody chains are located on preliminary single-gene vectors. These are thereafter cloned onto the respective multi-gene vector. An exemplary functional gene expression cassette consists of a CMV promotor, an Intron A, the 5′-untranslated region (5′-UTR) followed by the start codon ATG which is flanked by the Kozak sequences and the gene of interest (GOI) ending with an polyadenylation signal (polyA) [7], [8]. Cloning of vectors applies via restriction enzymes and ligation and/or the DNA assembly reaction.

Thus, expression vectors are cloned by the digestion of the gene cassettes out of the preliminary single-gene vectors and their subsequent ligation into the multiple cloning site (MCS) of the backbone vector, resulting in a multigene vector. Therefore, suitable restriction enzymes are selected according to the MCS. Restriction enzymes (REs) are catalytic enzymes hydrolyzing dsDNA at specific sequences. They cut the dsDNA strand along the palindromic pattern in the recognition site and create single-stranded overhangs called sticky ends [9]. When two fragments are cut with the same enzyme, the overhangs resulting in complementary single strands, which can attached by ligation. Enzymes cutting in the MCS are used and chosen in a manner, so that the individual fragments are ligated only in the correct arrangement.

Likewise, cloning via an assembly reaction is known. This cloning method allows for seamless assembly of multiple DNA fragments because of homology sequences at the end of each fragment. Recombination sites (R-sites) are 15-80 bp long sequences, surrounding the gene cassette of the GOI. Depending on the arrangement, each fragment has a specific R-site at the beginning and the end of the sequence, which is identical to the R-sites of the previous or subsequent fragment. Therefore, a cloning of the single genes in particular preliminary vectors containing the right R-sites is necessary. Afterwards, the genes flanked by the R-sites is cut out by restriction enzymes cutting next to the R-sites. Finally, the assembly of all fragments occurs in one-step. The exonuclease creates single-stranded 3′ overhangs that facilitate the annealing of fragments that share complementarity at the R-sites. The polymerase fills in gaps within each annealed fragment and the ligase fills gaps in the assembled DNA [10], [11]. FIG. 2 provides a schematic overview of the assembly reaction. Compared to RE cloning, the assembly of individual fragments via R-sites is already an improvement in efficiency and effort.

### DETAILED DESCRIPTION OF EMBODIMENTS OF THE INVENTION

The currently used methods for cloning of expression vectors all rely on the cloning of single-gene vectors that provide either the specific restriction enzyme or specific R-sites flanking the gene expression cassette of interest. Thus, for highly complex molecules a huge number of individual vectors, e.g. more than 20 single-gene vectors, are required to generate the final multi-gene expression vectors. This represents a time-consuming and labor-intensive process step. There is the need with increasing complexity of the molecules to be expressed that new time-, effort and resource-saving cloning strategies are provided, especially for creating multi-component vector systems. This task has been solved in the current invention!

In the current invention, an efficient cloning system for multi-gene expression vectors containing only a single cloning step without the need for the generation of preliminary single-gene vectors is reported. The cloning method according to the current invention is based on PCR-produced components (vector backbones, adaptors) and the antibody chains as supplied, as well as synthesized DNAblocks.

The invention is based, at least in part, on the finding that in order to link different expression cassettes in a single step without intermediate provision of single-gene vectors, adaptors spanning the termination sequence of the previous expression cassette and the promotor of the following expression cassette can advantageously be used.

Thus, each nucleic acid fragment used in the method according to the current invention is flanked by a recognition site for a restriction enzyme and a specific, unique recombination site (R-site).

The current invention is based, at least in part, on the finding that the flanking by a recognition site for a restriction enzyme and a specific, unique recombination site allows for direct cloning of multiple gene cassettes into the respective vector backbone. As this is done without intermediate single-gene vector cloning steps, it enables fast cloning of differing gene configurations. A further advantages of the modification of the nucleic acid fragments according to the current invention is the unique character of the R-site sequences, which provide new binding sites for primer in case of sequence variant analysis (SVA). A further advantage of the method according to the current invention is that the method according to the current invention reduces the required time and effort for gene synthesis. This is achieved by the generation and modification of the employed nucleic acid fragments via PCR.

In one example, the method according to the current invention was used to generate targeted integration front and back vectors, which together code for the different chains of a bispecific antibody.

### Exemplary Description of the Method According to the Current Invention

The following is an exemplary description of the method according to the current invention. This is chosen simply as an example. Any other vector system can be likewise produced, as the method according to the current invention is generally applicable.

In the following, the method according to the current invention is exemplified with a two-vector targeted integration system in which one part of the antibody genes are located on the front vector and the other on the back vector. The front and back vectors carry all required elements to survive selection pressure after integration into the landing site in the TI-host cell's genome.

Depending on the intended expression cassette configuration, the DNAblocks coding for the respective chains of the antibody are assembled with the method according to the current invention and integrated into basic vector backbones.

In the method according to the current invention, nucleic acid sequence adaptors (or short adaptors) are used, which contain the terminator of the previous DNAblock and the promotor sequence of the following, whereby the promotor/terminator sequence of the first/last DNAblock is located on the vector backbone. This ensures the correct arrangement of the DNAblocks. Each adaptor and DNAblock carries a recognition sequence for a restriction enzyme at its 5′-end and a unique R-site at its 3′-end. This enables the insertion of the individual gene expression cassettes according to intended configuration. In FIGS. 3A and 3B, the method is schematically illustrated for a bsAb with the configuration xLC-knob—LC-hole. Linearized backbones were generated by digestion of the respective basic vector with the restriction enzyme PspOMI. Likewise, any other restriction enzyme cleaving the backbone vector only once and in the intended region can be used. Then, the ligation of the cleavage sites (represented by scissors) of each fragment pair occurs in separate reactions. The first DNA-block is hence a part of the backbone, wherein the first adaptor is ligated with the second DNA-block, resulting in a longer fragment length. In the assembly reaction (represented by crossed lines), these ligated fragments are finally recombined with their corresponding fragment that shares the same R-site.

Thus, the cloning method according to the current invention is based on a modular system using pre-defined, generic parts (adaptors and front and back vector backbones) and molecule-specific parts (i.e. in this example the antibody chain sequences) that are modulated by PCR to contain required R-sites at the 3′-end. Depending on the desired gene configuration, these parts are connected and assembled to the final vectors using restriction enzyme as well as assembly cloning methods.

A vector backbone to be used in the method according to the current invention requires the sequence (promoter-5′-UTR) of the first expression cassette followed by a restriction enzyme recognition site. These sequences are placed immediately at the beginning of the integration site in the vector. Moreover, an R-site is located in front of the final polyA sequence at the end of the integration site in the vector backbone. Between the restriction enzyme recognition site and the final polyA sequence, a recognition sequence for a restriction enzyme that is different from the other is located. This cleavage site is used to close the backbone to from a circular vector.

Any vector can be adopted for use in the method according to the current invention. The backbone of the vector is used as the basic structure of the vector for use in the method according to the current invention. This backbone is extended with the required sequences, two DNA fragments synthesized via PCR, which are subsequently ligated in the backbone vector. The procedure is depicted schematically for the front vector backbone in FIG. 4. Compared to the front vector, the back vector backbone encodes the Lox sites LoxFAS and L2, respectively, and forms after ligation with fragment 1 and fragment 3 (via RE's BamHI, PspOMI and PacI) the targeted integration back vector backbone.

Generally, for the generation of the vector backbones to be used in the method according to the current invention, primer pairs were employed, having 3′-overhanging sequences comprising overhang sequences according to the inventive concept additionally to their unique binding site. For example, in order to produce fragment 1 of FIG. 4, a template was used comprising the sequences CMV-Intron A-5′-UTR-RE and was amplified with the respective primer pair. Fragment 2 and fragment 3 were likewise amplified from a plasmid carrying the polyA sequence. The PCR-generated fragments were analyzed using an analytical agarose gel electrophoresis (AGE).

All PCR-amplified fragments are flanked by RE recognition sites and can subsequently be integrated into the correct position of the vector backbone. Therefore, PCR samples were digested with the respective restriction enzyme (BamHI/PspOMI, PspOMI/PacI) and subsequently purified. To prevent self-ligation of the vectors, the backbones were digested with the RE's BamHI and PacI and afterwards dephosphorylated. Then, all digested inserts were ligated into the respective backbone vector. Chemical competent cells were transformed with the ligation products and spread on agar plates containing selection pressure. Sequencing of the respective sequence was used to verify the sequences identity of one of the clones of the correct backbone vectors. Finally, the vectors were enriched in a plasmid maxi preparation and used henceforth as basic vector backbones.

The invention is based, at least in part, on the finding that for efficient assembly of the gene fragments an RE of type IIB has to be used. Type IIB REs encompass BsaXI, CspCI, AloI, PpiI, PsrI, FalI, Bsp24I, BcgI, BpII, HaeIV, CjeI, CjePI, Hin4I, BaeI, AlfI, and BslFI (see, e.g., Tengs, T., et al., Nucl. Acids Res. 32 (2004) e121). These REs cleave both DNA strands at specified locations distant from their recognition sequences, i.e. these enzymes cut both strands on both sides of their recognition sequence and, thereby, arbitrary cut site, which are not palindromic/symmetric, are created [12]. The full cut site/recognition sequence of BsaXI as an exemplary RE of type IIB is displayed below in compact form, with the recognition sequence highlighted by shading.

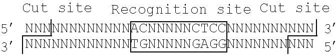

In order to ligate the fragments in the first cloning step, BsaXI as a type IIB enzyme was used as RE.

In one example of the method according to the current invention, front and back expression vectors were produced comprising the expression cassettes for the production of a T-cell bispecific antibody (TCB). The respective vectors had the expression cassette configuration kHC-xLC-xLC (front vector) and hHC-LC (back vector). Thus, the front vector is coding the gene expression cassettes of three antibody chains, the knob heavy chain and two crossed light chains wherein the back vector carries the hole heavy chain and light chain gene expression cassette. The cloning strategy for this example of the method according to the invention is outlined in FIGS. 5A and 5B.

In more detail, in the first step, the primers were designed to provide the adaptor and DNAblock fragments with the BsaXI recognition sites via PCR.

For the production of the different DNAblocks, single-gene vectors were used as templates. It has to be pointed out any other source is likewise suitable, e.g. genomic DNA, cDNA, etc. To extend the gene of the antibody chain with the appropriate R-site and BsaXI recognition sequence, primers were used carrying the respective additional sequences in their overhangs. In FIG. 6 the location of the primers for producing the various DNAblocks (solid lines) are depicted. The grey arrows represent the primers, with their binding site and their overhanging sequence. Below the template sequence, pictured as a solid line, the final PCR product is displayed with the expected length.

To ensure that the PCR-amplified fragments do not self-ligate, the sequences of the RE BsaXI were designed to carry either a symmetrical or palindromic cut site. As depicted below the BsaXI cut site at the 3′ sequence end was designed as three consecutive guanine bases.

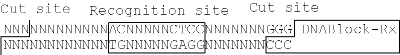

When the subsequent PCR-produced DNAblocks are digested with the BsaXI RE, the enzyme cleaves its recognition sequence from the entire fragment. This results in a CCC overhanging sequence at the 3′-end (antisense strand) of the DNAblock in form of a sticky end. Remaining arbitrary base pairs represented by the letter N were occupied randomly in a way to prevent the formation of secondary structures of the primer sequence. The use of a PCR temperature of 61° C. resulted in the most efficient amplification.

The cloning of the back vector was performed with the back basic vector backbone, HindIII-hHC-R11, R11-Adaptor-BsaXI and BsaXI-LC-R19. The TI front vector was cloned using the three DNAblocks HindIII-knob-R7, BsaXI-xLC-R9 and BsaXI-xLC-R1, which are connected via adaptors R7-Adaptor-BsaXI and R9-Adaptor-BsaXI. Therefore, the DNAblocks of both vectors were generated via PCR. A preparative AGE followed by gel extraction was performed. In FIG. 7, the various PCR-amplified DNAblocks are depicted in the agarose gel after electrophoresis. The bands running at expected sizes were extracted and purified from the agarose gel. The DNAblocks were then digested with the respective RE (HindIII, BsaXI) and purified.

To produce the adaptor fragments, a template was generated by isolating the CMV-Intron A-5′-UTR sequence from an existing vector by restriction digestion, followed by an AGE and finally the extraction from the gel. In order to produce adaptor fragments carrying a BsaXI recognition site the respective primer pairs were used.

According to the design of the BsaXI cleavage site in DNAblocks to connect the various DNAblocks a sticky end at the 3′ end of the adaptor sequence complementary to the CCC sticky end of the DNAblocks was introduced. Below the design of the BsaXI sequence within the adaptor fragment is shown, whereby the letter N represents an arbitrary base A, T, G, C.

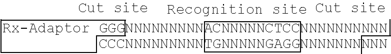

The subsequent PCR-produced adaptor is digested with the RE BsaXI, the enzyme cleaves its recognition sequence from the fragment and leaves a GGG overhanging sequence at the 3′-end of the adaptor sequence. This enables the ligation of adaptors and DNAblocks without the formation of by-products and with high efficiency, because neither adaptors nor DNAblocks are able to self-ligate.

Two different template concentrations per adaptor were tested. Therefore, the PCR reactions to generate the three adaptors were perfomed twice, once containing 20 ng of the template DNA and secondly containing 10 ng template DNA. As detectable from the respective agarose gel, the PCR reactions resulted in a inefficient production of the adaptor fragments (at 2069, 2072 and 2067 bp) and the amplification of non-specifc fragments above the target bands. Additionally, hybridized primers are visible in the lower part of the agarose gel, caused by the nearly 100 bp long forward primers.

It has been found that the PCR resulted in suitable amplification if a higher amount of template DNA and different forward and backward primer concentrations were employed.

FIG. 8 shows the PCR results performed with an amount of template DNA of 30 ng and two different primer concentrations for each adaptor fragment. In the first chamber within each adaptor (sample A, B, C) a defined amount of forward primer was used, whereby the amount used for the second gel chambers was half the amount of forward primer from the first gel chamber. The increase of the temple DNA together with a reduction of the amount of the forward primer resulted in an increased efficiency of adaptor amplification with the loss of hybridized primers as well as the reduction of unspecific fragments. Moreover, an additional improvement was achieved by employing less primer concentration. This resulted in a thicker band in each adaptor PCR (second gel chamber of each sample A, B, C).

Thus, it has been found that for producing the adaptor fragments the use of 30 ng template DNA in combination with the forward primer being used at half the amount as the reverse primer the adaptors can be amplified at suitable amount and quality. It has further been found that the appropriated annealing temperature is about 64.6° C.

The two PCR reactions per adaptor were separated via preparative AGE resulting in bright isolated bands for each PCR-amplified adaptor fragment at the expected sizes of 2069 bp for R7-Adaptor-BsaXI, 2072 bp for R9-Adaptor-BsaXI and 2062 bp for R11-Adaptor-BsaXI. The PCR-produced adaptor fragments running at the expected sizes were cut out and were extracted.

The multiple ligations of the linear fragments are the first step in the method according to the current invention. For the exemplary used antibody format (knob-xLC-xLC-hole-LC), three parallel ligations (labelled as scissors) result in the final front vector and two parallel ligations result the final back vector. The final front vector requires the ligation of the linear front basic vector backbone and the first DNAblock HindIII-knob-R7, the ligation of the first adaptor R7-Adaptor-BsaXI with the second DNAblock BsaXI-xLC-R9 and the ligation of the second adaptor R9-Adaptor-BsaXI with the last DNAblock BsaXI-xLC-R1. The final back vector requires the ligation of the digested back basic vector backbone with the first DNAblock HindIII-knob-R7 and the ligation of the R11-Adaptor-BsaXI with the second DNAblock BsaXI-LC-R19. For all ligations, the mass of the larger fragment was used to calculate the mass of the second fragment in a molar ratio of 1:1.

For ligation, the front basic vector backbone was digested with the RE's PspOMI and HindIII, dephosphorylated subsequently in the same reaction and finally purified via purification kit. After ligations, all samples were separated by a preparative AGE followed by the extraction of the correctly ligated fragments. The target ligation product backbone-front-knob-R7 was running at 8215 bp with the non-ligated linear backbone front running beneath at 6009 bp. The band of the non-ligated single DNAblock HindIII-knob-R7 runs at 2210 bp with the self-ligated DNAblock (R7-knob-knob-R7) running at 4420 bp. The ligation of the R7-Adaptor-BsaXI and the DNAblock BsaXI-xLC-R9 resulted in a bright, separated band (R7-Adaptor-xLC-R9) running at the correct size of 2913 bp. The bands beneath, are single non-ligated fragments R7-Adaptor-BsaXI (at 2038 bp) and the DNAblock BsaXI-xLC-R9 (at 878 bp), without any self-ligated fragments as by-products. The same is shown in FIG. 9C, where the framed band running at 2914 bp represents the target ligation product R9-Adaptor-xLC-R1 as a product of the ligation of R9-Adaptor-BsaXI (2041 bp) and the DNAblock BsaXI-xLC-R1 (876 bp) without self-ligation. In each gel chamber, two of the same ligation mixtures were unified and separated.

For ligation, the back basic vector backbone was digested with the RE's PspOMI and HindIII, subsequently dephosphorylated in the same reaction and finally purified via purification kit. After the backbone was ligated with the first DNAblock HindIII-hole-R11 and the adaptor R11-Adaptor-BsaXI was ligated with the second DNAblock BsaXI-LC-R19, the reactions were analyzed in a preparative AGE and the target ligation products were finally purified via gel extraction. As visible in Lane 10A, the ligation resulted in a bright isolated band for the target ligation product backbone-back—hole-R11 at the expected size of 7587 bp. The non-ligated linear backbone back is represented also by a bright isolated band at 6062 bp. At the size of 1528 bp the single non-ligated DNAblock HindIII-hole-R11 is visible, with the self-ligated fragment at 3056 bp (R11-hole-hole-R11). For the second ligation (Lane 10B) a bright isolated band appeared for the target ligation product R11-Adaptor-LC-R19 with the expected size of 2842 bp. The bands of the non-ligated single fragments runs at 2031 bp for R11-Adaptor-BsaXI and 814 bp for BsaXI-LC-R19. The bands of the target ligation products backbone-back—hole-R11 and R11-Adaptor—LC-R19, running at the expected sizes were cut out and finally extracted and purified from the gel slices.

In sum, the ligations of the newly generated fragments carrying the BsaXI cleavage sites are efficient and leads to a high yield of the target ligation products.

In the assembly reaction, the various ligated fragments are finally recombined with their corresponding fragment that shares the same R-site. Therefore, all extracted and purified ligation products for the corresponding vector were assembled in one single reaction.

The DNA amount for each assembly reaction were calculated with the aim of a total DNA amount of 0.2 pmol for all fragments. A molar ratio of 1:2 vector:insert were considered for this calculation.

After assembly, chemical competent bacteria cells were transformed with the TI vectors, followed by plasmid preparations of clones found on the selection plates. The isolated final vectors were then tested for correctness by control digestion as well as Sanger sequencing of plasmids containing a correct band pattern after an AGE.

For the assembly reaction of the final front vector, three fragments are required according to the configuration of the TCB, wherein the ligation products are backbone-front—knob-R7, R7-Adaptor—xLC-R9 and finally R9-Adaptor—xLC-R1. Each fragment ends with the initial R-site of the following fragment. All ligated fragments were unified in one assembly reaction. Afterwards, two types of chemical competent cells were transformed with the assembly products and spread on agar plates containing selection pressure. As a positive control, a predefined assembly control mixture was assembled in parallel and transformed into the cells, whereas an assembly and following transformation with water served as a negative control. After incubation overnight, clones have grown carrying a circular plasmid, which encodes the selection marker. The positive control plate was overgrown with over 1000 colonies, whereas on the negative control plate no colonies were found. The selection plate after the transformation of 5-alpha F'Iq competent E. coli bacteria resulted in 18 isolated, single colony-forming units (CFU). Less but thicker colonies were found on the plate after transformation of 10-beta competent E. coli bacteria.

A total of 10 clones of the transformations with assembled final front vectors were picked and cultivated for a plasmid preparation. A test digestion with three enzymes (HindIII, PvuI and AfeI) of the isolated plasmids followed by an AGE was performed to determine which clones carry the desired TI front vector containing all three assembled fragments in the correct arrangement. Of plate 1 (5-alpha F'Iq competent E. coli bacteria) clones showing five bands with the expected sizes of 4612 bp, 3346 bp, 2836 bp, 1642 bp and 1374 bp could be isolated. Of the enriched plasmids from plate 2 (10-beta competent E. coli bacteria) none of the three grown colonies show a correct band pattern.

Sequencing of the respective sequence was used to verify the sequences identity of one of the clones of the correct final front vectors. The sequencing result confirmed that the correct final font vector was generated via the new cloning method according to the current invention.

For the assembly reaction of the final back vector, the two ligated fragments are required according to the configuration of the TCB, wherein the ligation products are backbone-back—hole-R11 and R11-Adaptor—LC-R19. These were unified in one assembly reaction. Two different bacteria stains were transformed with the assembly product and spread on plates including selection pressure. The same positive and negative controls as for the final front vector were used. As expected, after incubation over 1000 colonies were found on the positive control plate and none on the negative control plate. The transformed 10-beta competent E. coli bacteria resulting again in less but thicker grown colonies. A total of 20 clones were picked to perform plasmid preparations.

In order to verify which clones carry the correctly assembled plasmid, the enriched plasmids were digested with the enzymes HindIII, EcoRI and PacI and analyzed in a preparative AGE. A clone is considered as correct, if four bands appear at the sizes 5668 bp, 2776 bp, 1433 bp, 422 bp. Nearly 90 percent of the tested transformants may carry a correct vector according to the obtained band pattern. Of the transformations of 5-alpha F'Iq competent E. coli bacteria, the control digestions of 7 out of 10 clones resulted in a correct band pattern at correct sizes. In the transformations of 10-beta competent E. coli bacteria, 9 out of 10 supposedly correct clones were found.

The sequences of the selected plasmids were then determined by Sanger sequencing and compared to the reference sequence. The alignment of the sequenced vector area with the reference sequence resulted in a 100 percent coverage of both sequences.

Compared to the 10-beta competent E. coli bacteria, the 5-alpha F'Iq competent E. coli bacteria are more efficient in the transformation process, leading to a greater number of colonies on the plate. This is particularly advantageous if more than one insert is involved in the assembly reaction. Therefore, it is recommendable to use the 5-alpha F'Iq competent E. coli bacteria for subsequent cloning of TI vectors consisting of three or more genes of antibody chains in form of DNAblocks.

### Summary and Outlook

Herein a modular and efficient method for cloning multigene plasmids containing only a single cloning step without the use of preliminary single-gene vectors is reported.

In the first cloning step, PCR-produced DNAblocks carrying genes for different antibody chains are ligated with the respective adaptors, which serve as connectors of the different DNAblocks.

The method is based on the ligation of the individual fragments, i.e. backbone, DNAblocks and adaptors, via the RE cleavage sites located at one end of each fragment. It has been found that REs have to be used that do not generate a symmetric or palindromic cleavage site. Thereby the generation of self-ligation by-products can be reduced or even eliminated.

In the second step, the ligated fragments of the first step are assembled in the correct arrangement via the unique R-sites located at one end of each backbone, DNAblock and adaptor fragment.

This new strategy results in a modular and efficient method, which allows direct cloning of expression cassettes into the respective backbone without intermediate cloning steps and enable fast cloning of variable gene configurations of diverse antibody formats. This new cloning method according to the current invention provides considerable advantages in terms of time, work labor and costs.

### COMPARATIVE EXAMPLE

In a comparative example, front and back vectors for targeted integration encoding a bispecific antibody were cloned via the cloning method according to the current invention but a Type IIP RE, i.e. HindIII, with a symmetric target and cleavage sites was used. The BsAb consists of two heavy chains (knob and hole) and two light chains, whereby one of them is crossed. The front vector (FIG. 3A) encodes the crossed light chain (xLC) and knob heavy chain (kHC), and the back vector (FIG. 3B) carries the genes of light chain (LC) and the hole heavy chain (hHC) (xLC-kHC—LC-hHC).

In the first step, primer pairs are employed, having 3′-overhanging sequences whereby the overhang sequences are in addition to the unique binding sites. In order to produce fragment 1 of FIG. 4, a template was used carrying the sequences CMV-Intron A-5′-UTR-HindIII and was amplified with the primer pair oSA105/106. Fragment 2 and fragment 3 were amplified from a source DNA carrying the polyA sequence by using the primer pairs oSA107/108 and oSA109/108, respectively. FIG. 11 depicts the binding sites and sequence overhangs of the primers, the templates and the amplified PCR products with their anticipated lengths.

The PCR-generated fragments were analyzed using an analytical agarose gel electrophoresis (AGE). Compared to the DNA-marker all fragments sizes complied with the expected fragment lengths of 1663 bp for fragment 1, 421 bp for fragment 2 and 413 bp for fragment 3.

All PCR-amplified fragments are flanked by RE recognition sites and can subsequently be tempted to be integrated into the vector backbone. Therefore, PCR samples were digested with the respective restriction enzyme (BamHI/PspOMI, PspOMI/PacI) and subsequently purified. To prevent self-ligation of the vectors, the backbones were digested with the RE's BamHI and PacI and afterwards dephosphorylated. Then, all digested inserts were ligated into the respective backbone. Chemical competent cells were transformed with the ligation products and spread on agar plates containing selection pressure. Sequencing of the respective sequence was used to verify the sequences identity of one of the clones of the correct TI basic vectors. Finally, the vectors were enriched in a plasmid preparation and used as vector backbones.

Thereafter, the DNAblocks flanked by HindIII recognition site and the respective R-site as well as the adaptors were produced via PCR, which serve as connectors for the individual DNAblocks. According to the method the individual fragments were ligated, i.e. the vector backbone with the first DNAblock and the adaptor with the second DNAblock via ligation of the HindIII cleavage sites. Thereafter, the ligated fragments were assembled in a single reaction. For the production of the individual DNAblocks, vectors carrying the respective gene of the antibody chains were used as templates for the PCR. It has to be pointed out any other source is likewise suitable, e.g. genomic DNA, cDNA, etc. To extend the template sequences with the sequences of HindIII cleavage sites and individual R-sites, specific primer pairs were used. The location of the primers (blue thick arrows) and the final amplified product (solid blue line) are depicted in FIG. 12. It has been confirmed that an annealing temperature of 61° C. is for all DNAblocks the most efficient resulting in isolated and light bands in AGE.

Thus, the DNAblocks HindIII-xLC-R15 and HindIII-knob-R1 were produced by PCR. In FIG. 13 single, bright bands appeared at the expected sizes of 1496 bp for the DNAblock HindIII-xLC-R1 (FIG. 13—left gel) and 861 bp for the DNAblock HindIII-xLC-R15 (FIG. 13—right gel).

For producing the adaptors, the template was generated by isolating the sequence CMV-Intron A-5′-UTR from an existing vector using the RE BstEII. It has to be pointed out any other source is likewise suitable, e.g. genomic DNA, cDNA, etc. The different fragments were separated by an AGE and then extracted from the gel. Specific primer pairs were used, having preferably one binding site in the template fragment. The overhanging sequence of the forward primer encodes sequences for the unique R-site, whereby the reverse primer encodes the cleavage site of HindIII in the binding site. It has been confirmed that the most efficient amplification of the fragment sequence occurs at 64.6° C.

Thus, the R15-Adaptor was produced by PCR. An analytically AGE was performed with a part of the PCR reaction product (FIG. 14). Bright bands, without any by-products appeared at the expected size of 2045 bp. The remaining PCR reaction mix was digested with the HindIII RE and purified.

The first cloning step was the ligation of the various fragments. For this antibody format and configuration, two simultaneously ligations are necessary. The ligation of the front vector backbone with the first DNAblock (ligation 1) and the ligation of the adaptor and the final DNAblock (ligation 2). In both ligations 500 ng of the larger fragment was applied and the mass of the second fragment was calculated in a 1:1 molar ratio.

First, for ligation 1, the front vector backbone was digested with PspOMI and HindIII, parallel dephosphorylated in the same reaction and then purified using a commercial purification kit according to the instructions of the manufacturer. After ligation, all samples were separated by a preparative AGE to extract the correct ligated fragments. In the first gel chamber (FIG. 15) four isolated bands appear. The two faint bands running in the lower part of the gel could be assigned to the non-ligated fragment R15-xLC-HindIII (856 bp) and to the self-ligated fragment R15-xLC-xLC-R15 (1712 bp). The target ligation product (backbone-front—xLC-R15) is the largest fragment and thus the top bright band at 6857 bp, with the non-ligated linear backbone front just beneath. The bands of ligation 2 in the second gel chamber are as follows: the three main products are R15-Adaptor-HindIII (2038 bp) and HindIII-knob-R1 (2982 bp) as non-ligated single fragments and the self-ligated HindIII-knob-R1 fragment (R1-knob-knob-R1); the target ligation product (R15-Adaptor—knob-R1) is represented by a faint band at 3525 bp. Thus, ligation 2 using a Type IIP RE, i.e. HindIII, with a symmetric target and cleavage sites is not efficient as shown by the many side-products.

Without being bound by this theory, it is assumed that the high rate of self-ligations is caused by the symmetric cut site of the HindIII RE. After cutting the palindromic recognition site, a 5′ AGCT overhang occurs which is complementary to the overhang of the same fragment in a double-turned orientation.

Furthermore, without being bound by this theory, a possible explanation for the bands that appear in ligation 2, which cannot be assigned to a defined fragment, is that in PCR for the production of the fragments, the primers additionally bind in the R-site and, thus, fragments of different sizes were amplified. In the thereafter-following ligation, the unspecific PCR fragments result in a variety of ligated fragments of different sizes. This theory would also explain the smearing that appears along the gel chamber.

Adaptor and DNAblock ligation was repeated and optimized several times (data not shown), whereby also each ligation was inefficient and by-products were generated. Optimization experiments were different PCR templates, heating before PCR, different ligases and ligation times.

Summarizing the result of this comparative example, the use of a type IIP endonuclease, i.e. the nature of the HindIII cut sites, resulted in a symmetric cleavage site, which in turn allowed DNAblocks and adaptors to self-ligate.

Consequently, the required amount of target ligation product for the later assembly reaction could not be achieved.

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Scheme of a two-plasmid RMCE strategy involving the use of three RRS sites to carry out two independent RMCEs simultaneously.

FIG. 2 Schematic DNA assembly reaction.

FIGS. 3A and 3B Cloning strategy according to the current invention exemplified by the specific antibody xLC-knob—LC-hole.

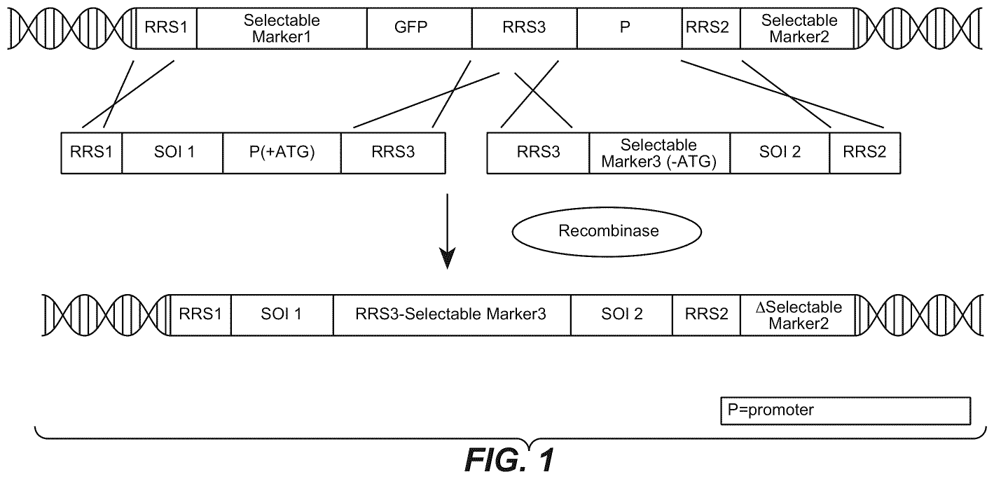

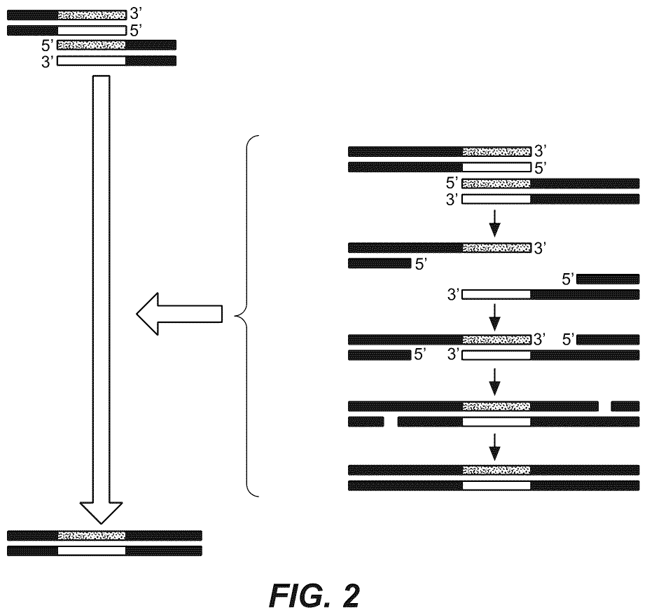

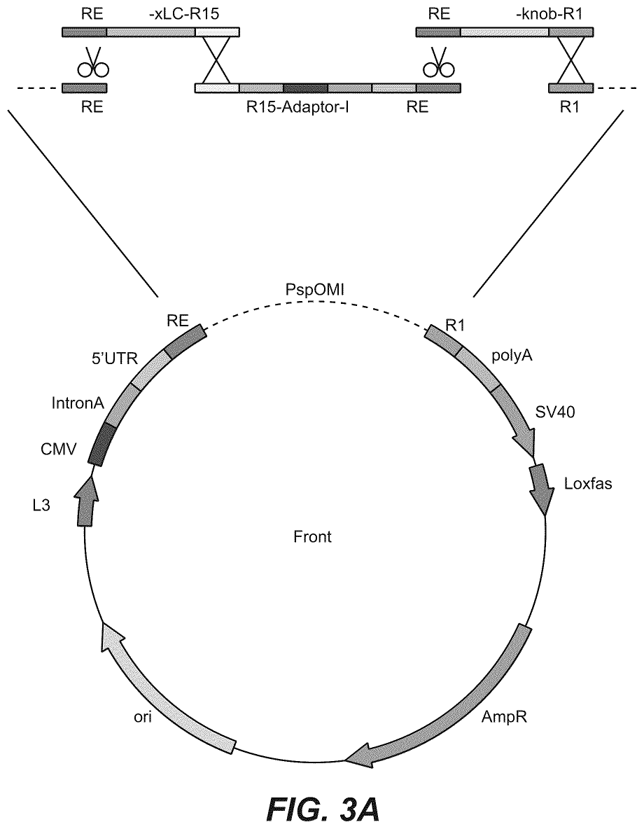

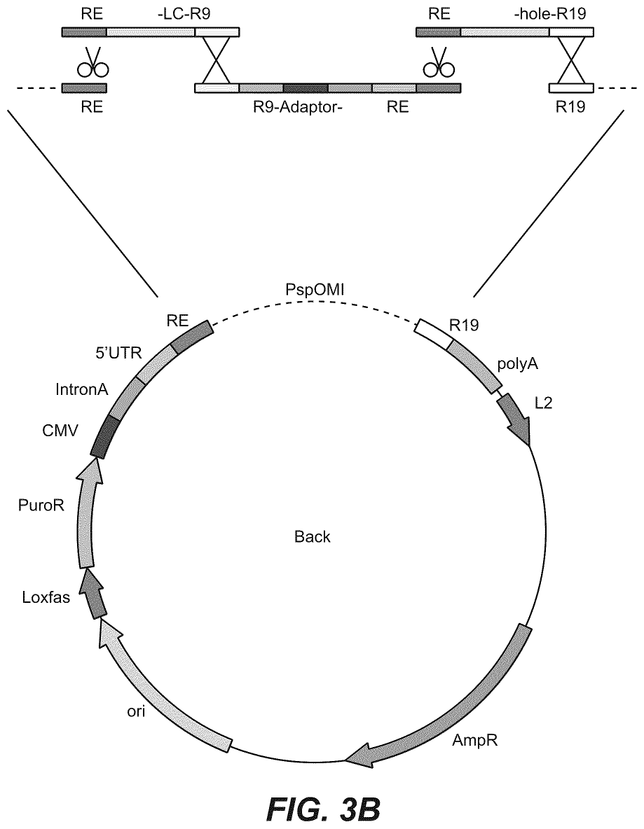

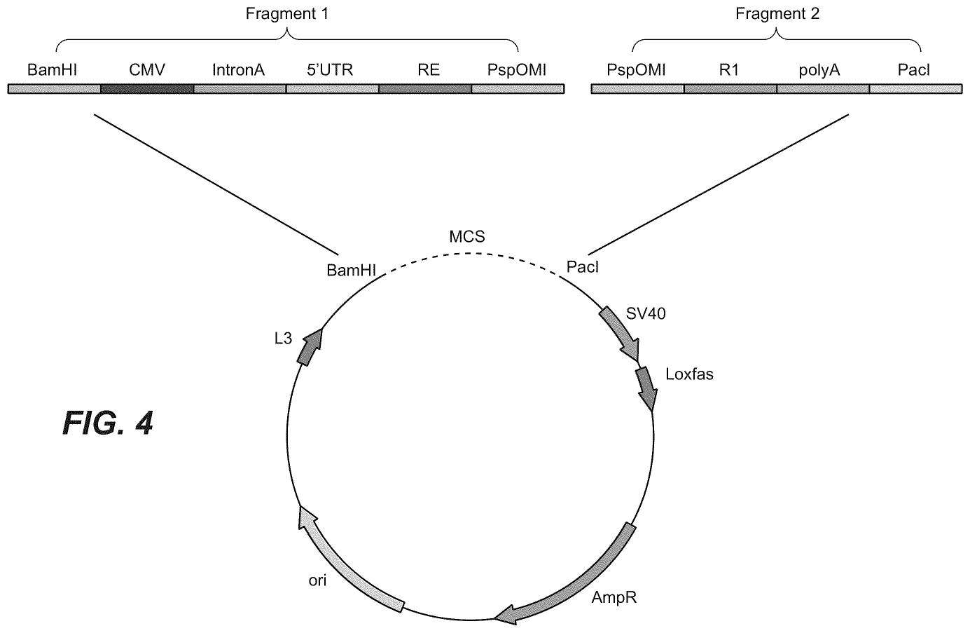

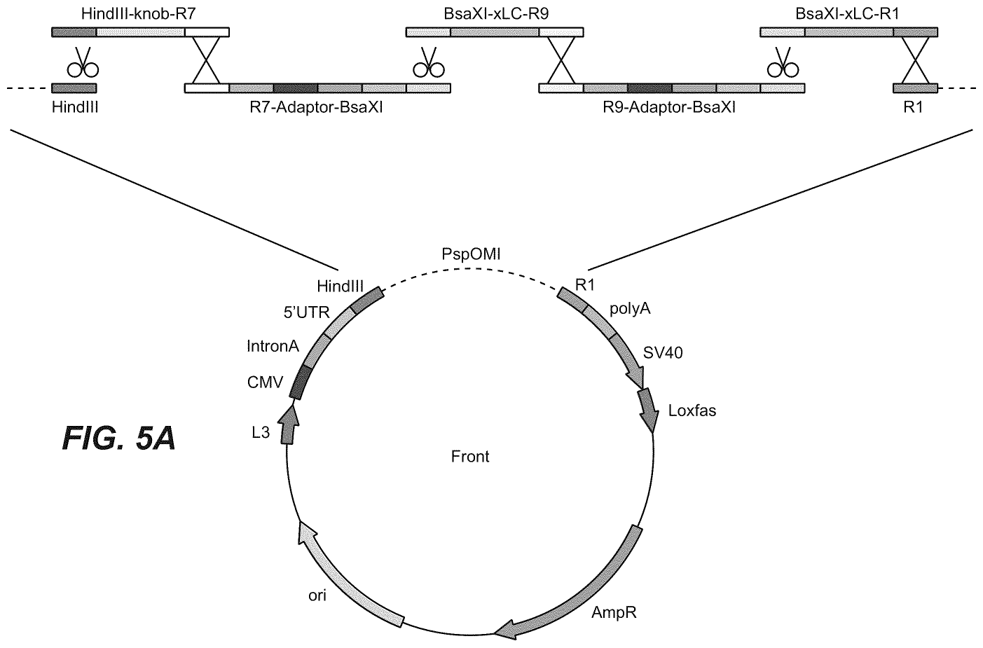

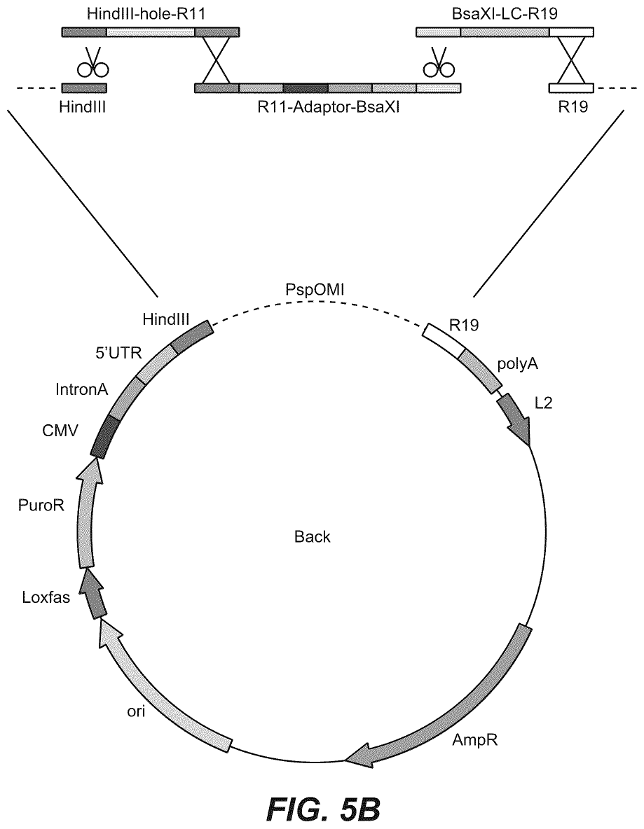

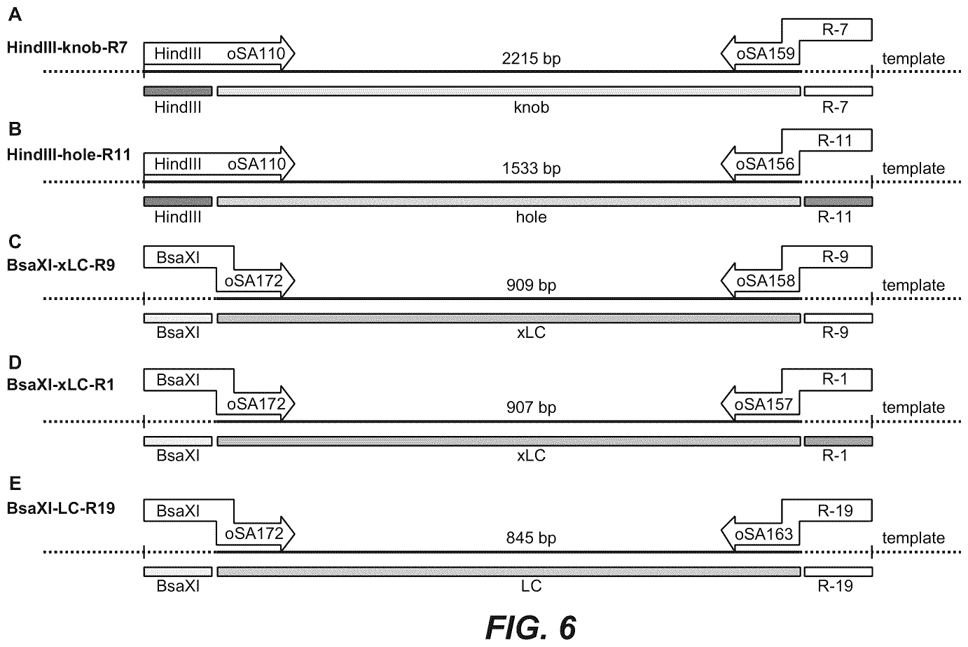

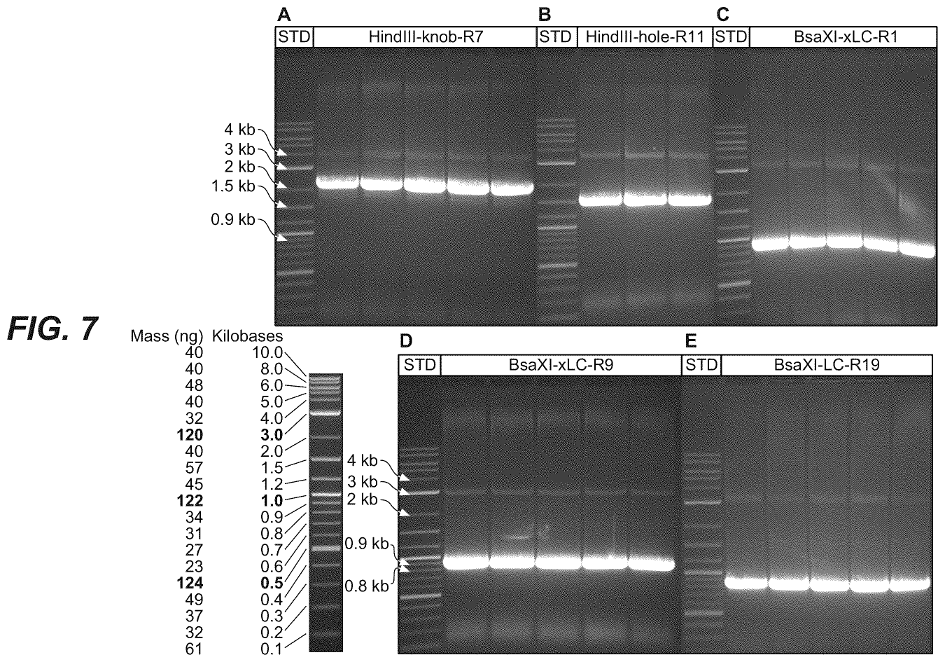

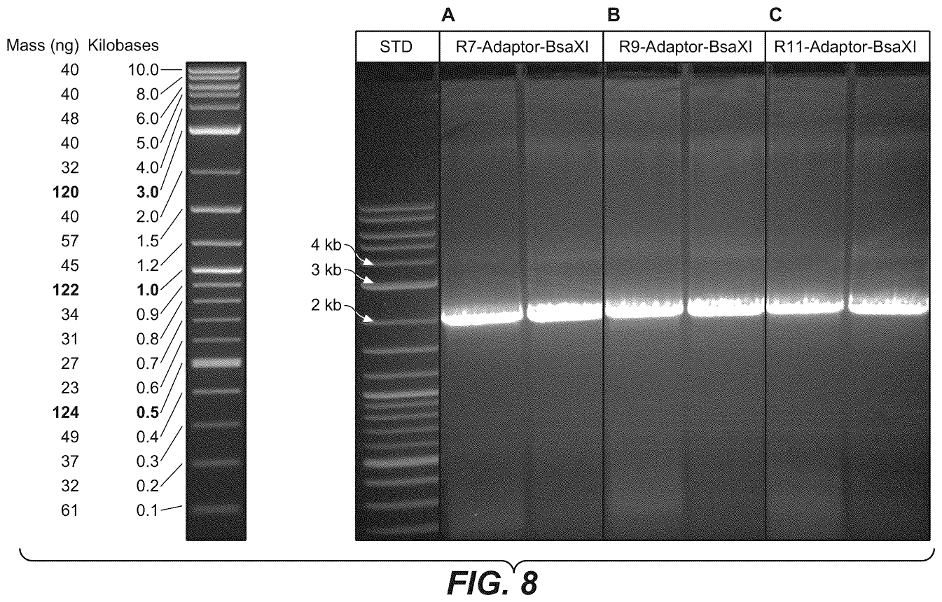

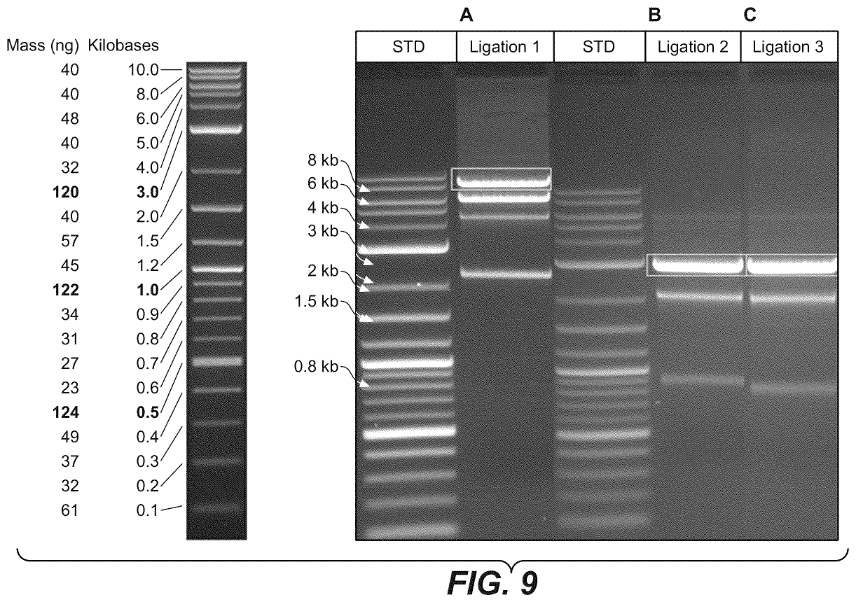

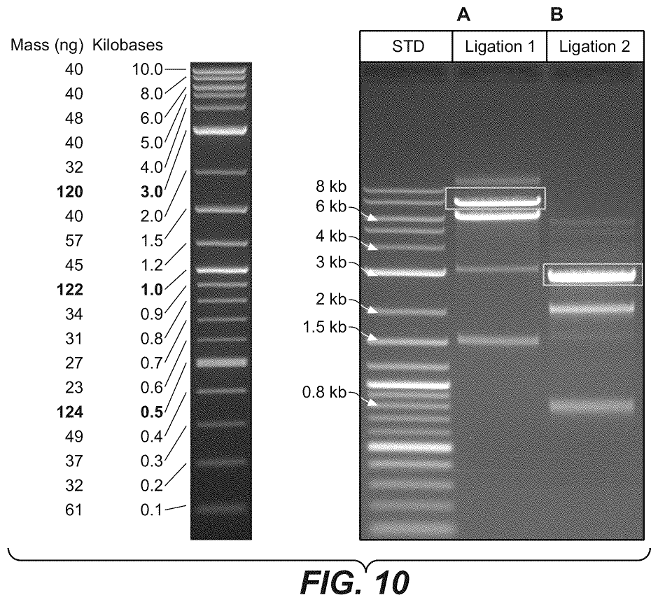

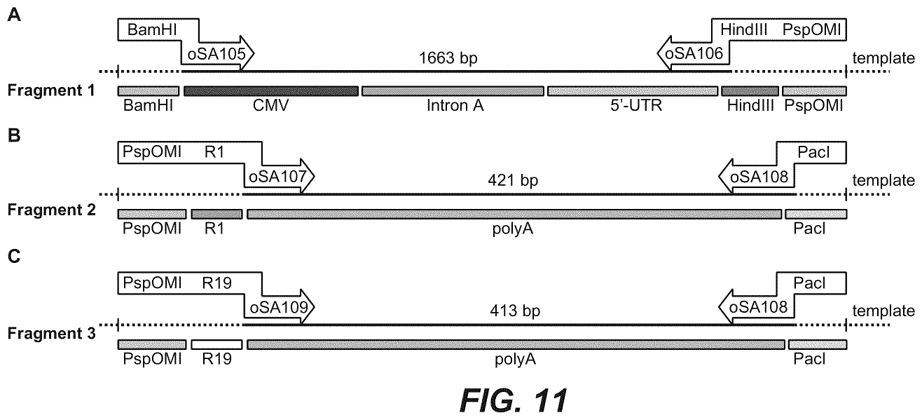

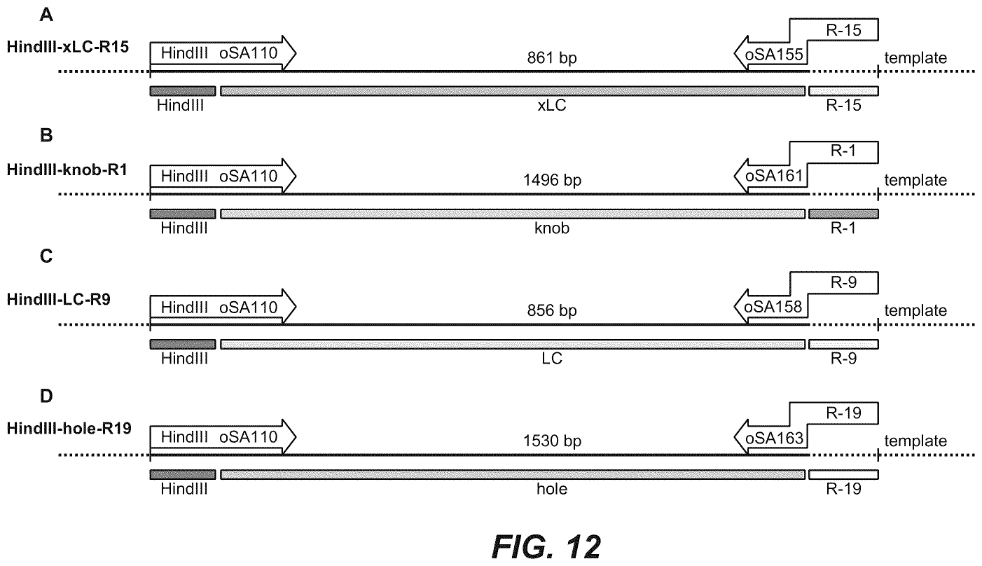

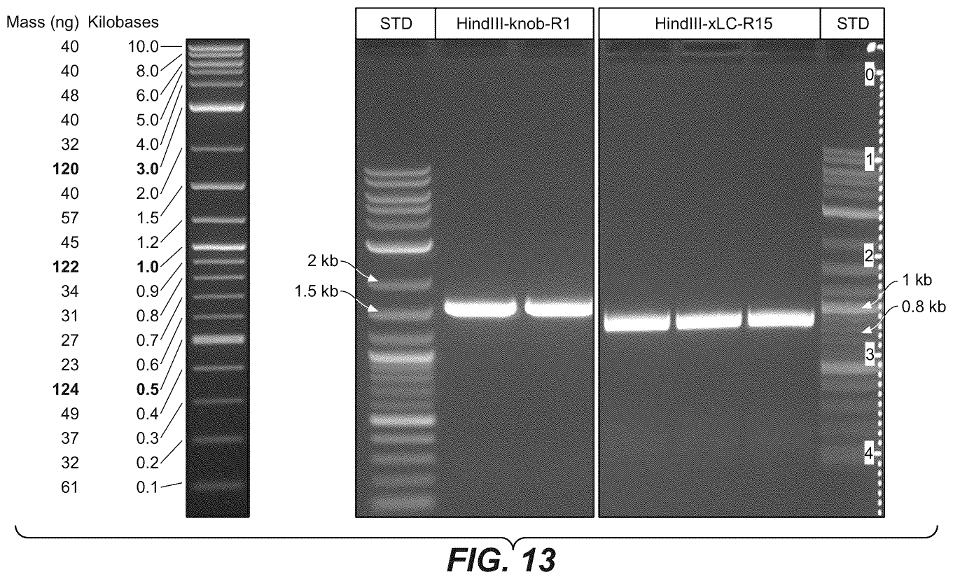

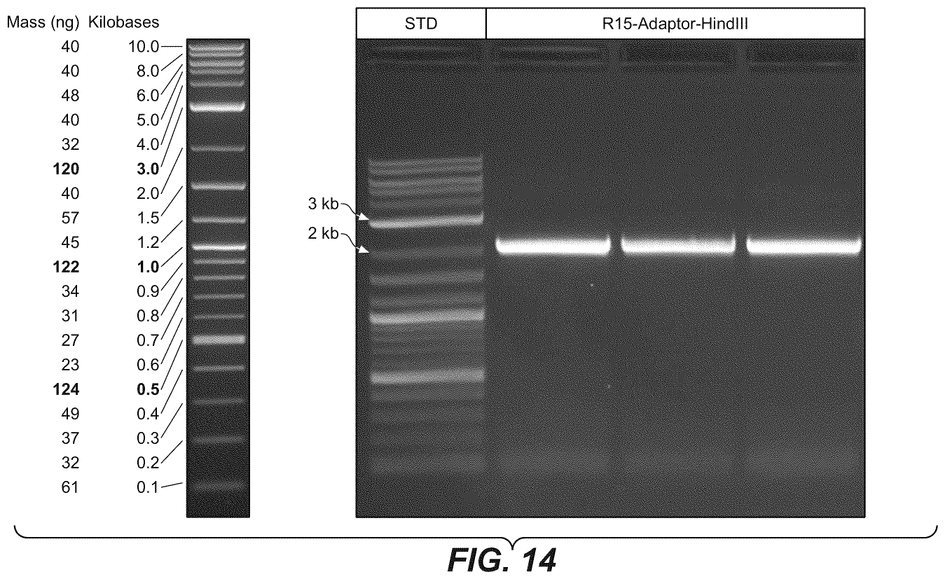

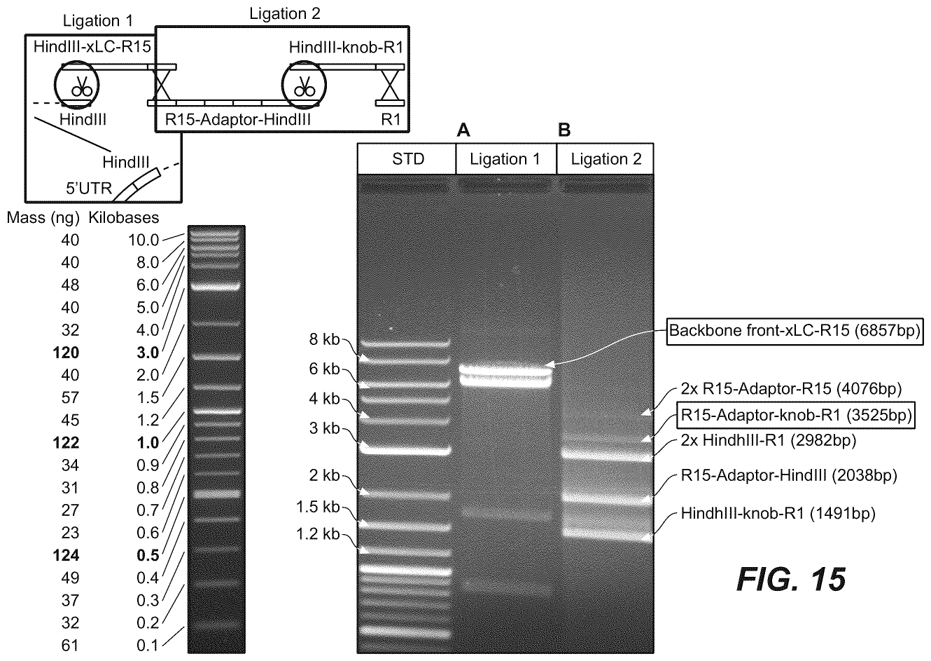

3A) TI front vector backbone carrying the initial sequence of the first DNAblock and the terminal polyA sequence of the last DNAblock. The vector is opened via PspOMI and a second RE, exposing the second RE's cleavage site and the R1-site. In the first step second RE's cleavage sites (labelled with scissors) are ligated in separate reactions. Secondly, the ligated two fragments are assembled together (labelled as crossed lines).

3B) TI back vector backbone carrying also initial and terminal sequences of DNA blocks and is also opened via PspOMI and a second RE, resulting in an exposed second RE's cleavage site and R19-site. The cloning procedure is the same for both vectors.

FIG. 4 Design and restriction cloning of vector backbone. Fragment 1 is coding for the initial sequence of the gene expression cassette from the first antibody chain and is used in the example for both targeted integration vector backbones. Fragment 1 and 2 are only distinguished by their R-site sequence. The front vector backbone is composed of the basic front vector backbone and the PCR-generated fragments 1 and 2. The vector backbone is carrying a multiple cloning site (MCS), which is flanked by two Lox sites, L3 and LoxFAS, respectively, and the RE recognition sites BamHI and PacI for subsequent ligation with the fragments.

FIGS. 5A and 5B Cloning strategy for the exemplary TCB knob-xLC-xLC—hole-LC.

5A) front basic vector backbone carrying the initially sequence of the first DNAblock and the terminal polyA sequence of the last DNAblock is opened via RE PspOMI, exposing the HindIII cleavage site and the R1-site. In the first step RE cleavage sites (labelled with scissors) are ligated in separate reactions. Secondly, the ligated three fragments are assembled together (labelled as crossed lines).

5B) back basic vector backbone carrying initial and terminal sequences of DNA blocks is opened via PspOMI, resulting in an exposed HindIII cleavage site and R19-site.

FIG. 6 Design and application of primers for DNAblock production and corresponding binding sites, overhangs, template, PCR product and expected fragment length.
A) HindIII-knob-R7 was amplified from the single-gene vector by using the primer pair oSA110/159.
B) HindIII-hole-R11 was amplified from the single-gene vector by using the primer pair oSA110/156.
C) BsaXI-xLC-R9 was amplified from the single-gene vector by using the primer pair oSA172/158.
D) BsaXI-xLC-R1 was also amplified from the single-gene vector by using the primer pair oSA172/157.
E) BsaXI-LC-R19 was amplified from the single-gene vector by using the primer pair oSA172/163.

FIG. 7 AGE of PCR-produced DNAblocks. Each PCR reaction was set in a volume of 50 μL containing 10 ng template and 2 μL of each primer (10 μM). All DNAblocks are represented as bright bands at the expected sizes of A) 2215 bp for DNAblock HindIII-knob-R7; B) 1533 bp for DNAblock HindIII-hole-R11; C) 907 bp for DNAblock BsaXI-xLC-R1; D) 909 bp for DNAblock BsaXI-xLC-R9; and E) 845 bp for DNAblock BsaXI-LC-R19. The expected fragments are compared to the 1 kb-plus-DNA-marker.

FIG. 8 Preparative AGE of PCR-generated adaptors amplified with two different forward primer concentrations.

Each adaptor was amplified with 30 ng template DNA and once with 1 μL forward primer (10 μM) (first chamber of each sample A, B, C) and secondly 0.5 μL forward Primer (second chamber for of each sample A, B, C) wherein the reverse primer was constant adjusted to 1 μL. The expected fragment sizes are A) 2069 bp for R7-Adaptor-BsaXI; B) 2072 bp for R9-Adaptor-BsaXI; and C) 2062 bp for R11-Adaptor-BsaXI and were compared to the 1 kb-plus-DNA-marker on the left site.

FIG. 9 Ligations of the linear DNA fragments of the front vector. Each ligation was performed with 500 ng of the larger fragment and the calculated amount of the respective second fragment in a molar ratio of 1:1. In each gel chamber, two of the same ligation mixtures were unified. All bands were compared to the 1 kb-plus-DNA-marker on the left site:
A) the blue framed target ligation product runs at 8215 bp, the non-ligated backbone at 6009 bp, the DNAblock HindIII-knob-R7 at 2210 bp with the self-ligated fragment at 4420 bp;
B) the blue framed target band runs at 2913 bp, the single non-ligated R7-Adaptor-BsaXI at 2038 bp and the non-ligated DNAblock BsaXI-xLC-R9 at the size of 878 bp; C) the target band runs at 2914 bp, the non-ligated fragment R9-Adaptor-BsaXI at 2041 bp and the DNAblock BsaXI-xLC-R1 at 876 bp.

FIG. 10 Ligations of the linear DNA fragments of the back vector. Each ligation was performed with 500 ng of the larger fragment and the calculated amount of the respective second fragment in a molar ratio of 1:1. All expected bands were compared to the 1 kb-plus-DNA-marker on the left site:
A) The blue framed target ligation product backbone-back—hole-R11 runs at 7587 bp whereas the non-ligated backbone back runs at 6062 bp beneath. The single non-ligated DNAblock HindIII-hole-R11 runs at 1528 bp with the self-ligated fragment at 3056 bp;
B) The blue framed target ligation product R11-Adaptor—LC-R19 runs at 2842 bp and the single non-ligated fragments R11-Adaptor-BsaXI runs at 2031 bp and BsaXI-LC-R19 at 814 bp.

FIG. 11 Design and application of primers for fragment production and corresponding binding sites, overhangs, template, PCR product and expected fragment length.
A) Fragment 1 was amplified from a template coding for the CMV-Intron A-5′-UTR sequence using the primer pair oSA105/106.
B) Fragment 2 was amplified from a template carrying the polyA sequence by using the primer pair oSA107/108.
C) Fragment 3 was amplified from the same template as Fragment 2 by using the primer pair oSA109/108.

FIG. 12 Design and application of primers for fragment production and corresponding binding sites, overhangs, template, PCR product and expected fragment length.
A) HindIII-xLC-R15 was amplified from the single-gene vector by using the primer pair oSA110/155.
B) HindIII-knob-R1 was amplified from the single-gene vector by using the primer pair oSA110/161.
C) HindIII-LC-R9 was amplified from the single-gene vector by using the primer pair oSA110/158.
D) HindIII-hole-R19 was amplified by using the single gene vector by using the primer pair oSA110/163.

FIG. 13 Analytical AGE of PCR-produced DNAblocks. The expected fragments are compared to the 1 kb-plus-DNA-marker on the left site.
left gel: the DNAblock HindIII-knob-R1 runs at the expected size of 1496 bp.
right gel) the DNAblock HindIII-xLC-R15 runs at the expected size of 861 bp.

FIG. 14 Analytical AGE of the PCR-produced R15-Adaptor-HindIII. Each PCR reaction was set in a volume of 50 μL containing 10 ng template and 2 μL of each primer (10 μM), whereby a small volume of the reaction mix was used for the analytical AGE. All DNAblocks are represented as bright bands at the expected sizes of 2045 bp compared to the 1 kb-plus-DNA-marker on the left site.

FIG. 15 Preparative AGE of the Ligations 1 and 2. Ligation 1 represents the ligation of the digested TI front basic vector with the HindIII-xLC-R15 DNAblock whereas ligation 2 is the ligation of the R15-Adaptor-HindIII and the second DNAblock HindIII-knob-R1. The expected fragments are compared to the 1 kb-plus-DNA-marker.
A) The target ligation product of ligation is Backbone front-xLC-R15 at the expected size of 6857 bp. The non-ligated linear TI front basic vector runs at 6009 bp. The non-ligated single HindJJJ-xLC-R15 runs at 856 bp wherein the self-ligated fragment runs at 1712 bp.
B) The target ligation product R15-Adaptor-knob-R1 runs at the expected size of 3525 bp and the non-ligated HindIII-knob-R1 runs at 1491 bp with the self-ligated fragment at 2982 bp. The single non-ligated fragment R15-Adaptor-HindIII runs at 2038 bp with the self-ligated fragment at 4076 bp.

### Description of the Abbreviations

5′-UTR 5′-untranslated region
AGE Agarose gel electrophoresis
Amp Ampicillin
att B Attachment site B
BsAbs Bispecific antibodies
CLD Cell line development
dNTP Deoxyribonucleotide triphosphate
dsDNA double stranded DNA
GFP Green fluorescent protein
GOI Gene of interest
HCL Host cell line
HyTK Hygromycin-thymidine-kinase
KiH Knob into hole
MCS Multiple cloning site
LB Luria-Bertani
RE's Restriction enzymes
RMCE Recombinase-mediated cassette exchange
R-site Recombination site
TCB T cell bispecific antibodies
TCR T cell receptor
TI Targeted Integration

### Description of the Sequences

| Primer | | |
|---|---|---|
| name | Nucleotide sequence | Description |
| oSA096 | GATCATAAGCTTCCTCTGTGTTCAGTG | Reverse primer of |
| | CTGATC (SEQ ID NO: 01) | adaptors |
| oSA097 | GCTTCCGTCGGATTTGCAAGACTCGC | Forward primer R1- |
| | GAGGATACGTGTACGAAACCAGAAGA | Adaptor-HindIII |
| | CGTATGCACTAGGTCAACAATGAAGC | |
| | GGCCGTTCTAGTTGCCAG (SEQ ID NO: | |
| | 02) | |
| oSA098 | GCTTCCTAACAGCGCCGGTGGGAGGG | Forward primer R7- |
| | TAATCAGAAGACTCTATCGCGACGCT | Adaptor-HindIII |
| | ATCGGGTCGTATTATAGGATGAAGCG | |
| | GCCGTTCTAGTTGCCAG (SEQ ID NO: | |
| | 03 | |
| oSA099 | TCGCCAGGATGCCGGTCAGCATATAT | Forward primer R9- |
| | CGATACTCAAGGCAGGTCAATTCGCA | Adaptor-HindIII |
| | CTGTGAGGGTCACATGGGCGTTTGGC | |
| | ACGGCCGTTCTAGTTGCCAG (SEQ ID | |
| | NO: 04) | |
| oSA100 | ACCGACCTGGGTTCGGCACTGTGGGC | Forward primer R11- |
| | AGTGTGAGGTATTGGCAGACGCCCAG | Adaptor-HindIII |
| | TGCCGAACAACACCTGACGGCCGTTC | |
| | TAGTTGCCAG | |
| | (SEQ ID NO: 05) | |
| oSA101 | GCTACTTGATGTCTTGCGACGTTCTTA | Forward primer R15- |
| | GAGATGGACGAAATGTTTCGCGACCC | Adaptor-HindIII |
| | AGGATGAGGTCGCCCTAGGCCGTTCT | |
| | AGTTGCCAG | |
| | (SEQ ID NO: 06) | |
| oSA102 | ATCGGTGGGAGTATTCAACGTGATGA | Forward primer R19- |
| | AGACGCTGGGTTCACGTGGGAATGGT | Adaptor-HindIII |
| | GCTTCTGTCCTAACAGGCGGCCGTTCT | |
| | AGTTGCCAG | |
| | (SEQ ID NO: 07) | |
| oSA105 | GATCATGGATCCGTTGACATTGATTAT | Forward primer, |
| | TGACTAG (SEQ ID NO: 08) | fragment1 |
| oSA106 | GATCATGGGCCCAAGCTTCCTCTGTGT | Reverse primer, |
| | TCAGTGCTG (SEQ ID NO: 09) | fragment1 |
| oSA107 | GATCATGGGCCCGCTTCCGTCGGATTT | Forward primer, |
| | GCAAGACTCGCGAGGATACGTGTACG | fragment2 |
| | AAACCAGAAGACGTATGCACTAGGTC | |
| | AACAATGAAGCGGCCGTTCTAGTTGC | |
| | CAG (SEQ ID NO: 10) | |
| oSA108 | GATCATTTAATTAAGACTGACGCGTC | Reverse primer, |
| | CTCAAAAAT (SEQ ID NO: 11) | fragment2, fragment3 |
| oSA109 | CTGGCAACTAGAACGGCCGCCTGTTA | Forward primer, |
| | GGACAGAAGCACCATTCCCACGTGAA | fragment3 |
| | CCCAGCGTCTTCATCACGTTGAATACT | |
| | CCCACCGATGGGCCCATGATC (SEQ ID | |
| | NO: 12) | |
| oSA110 | GAGGAAGCTTGCCGCCA | Forward primer, |
| | (SEQ ID NO: 13) | HindIII-DNAblock-Rₓ |
| oSA155 | TAGGGCGACCTCATCCTGGGTCGCGA | Reverse primer, |
| | AACATTTCGTCCATCTCTAAGAACGTC | DNAblock HindIII- |
| | GCAAGACATCAAGTAGCTATTCAACT | xLC-R15 |
| | AATGGCTTATGTACGG (SEQ ID NO: 14) | |
| oSA156 | GTCAGGTGTTGTTCGGCACTGGGCGT | Reverse primer, |
| | CTGCCAATACCTCACACTGCCCACAG | DNAblock BsaXI- |
| | TGCCGAACCCAGGTCGGTTATTCAAC | hole-R11 |
| | TAATGGCTTATGTACGG (SEQ ID NO: | |
| | 15) | |
| oSA157 | GCTTCATTGTTGACCTAGTGCATACGT | Reverse primer, |
| | CTTCTGGTTTCGTACACGTATCCTCGC | DNAblock BsaXI- |
| | GAGTCTTGCAAATCCGACGGAAGCTA | xLC-R1 |
| | TTCAACTAATGGCTTATGTACGG (SEQ | |
| | ID NO: 16) | |
| oSA158 | GTGCCAAACGCCCATGTGACCCTCAC | Reverse primer, |
| | AGTGCGAATTGACCTGCCTTGAGTAT | DNAblock HindIII- |
| | CGATATATGCTGACCGGCATCCTGGC | LC-R9/BsaXI-xLC-R9 |
| | GATATTCAACTAATGGCTTATGTACG | |
| | G (SEQ ID NO: 17) | |
| oSA159 | GCTTCATCCTATAATACGACCCGATA | Reverse primer, |
| | GCGTCGCGATAGAGTCTTCTGATTACC | DNAblock BsaXI- |
| | CTCCCACCGGCGCTGTTAGGAAGCTA | knob-R7 |
| | TTCAACTAATGGCTTATGTACGG (SEQ | |
| | ID NO: 18) | |
| oSA161 | GCTTCATTGTTGACCTAGTGCATACGT | Reverse primer, |
| | CTTCTGGTTTCGTACACGTATCCTCGC | DNAblock HindIII- |
| | GAGTCTTGCAAATCCGACGGAAGCTA | knob-R1 |
| | TTCAACTAATGGCTTATGTACGG (SEQ | |
| | ID NO: 19) | |
| oSA163 | GCCTGTTAGGACAGAAGCACCATTCC | Reverse primer, |
| | CACGTGAACCCAGCGTCTTCATCACG | DNAblock HindIII- |
| | TTGAATACTCCCACCGATTATTCAACT | hole-R19/BsaXI-LC- |
| | AATGGCTTATGTACGG | R19 |
| | (SEQ ID NO: 20) | |
| oSA168 | CAACGGCGGATGGAGTAACAGTTCAT | Reverse primer |
| | ATCTTCCCCCTCTGTGTTCAGTGCTGA | |
| | TC (SEQ ID NO: 21) | |
| oSA169 | GCTTCCTAACAGCGCCGGTGGGAGGG | Forward primer, R7- |
| | TAATCAGAAGACTCTATCGCGACGCT | Adaptor-BsaXI |
| | ATCGGGTCGTATTATAGGATGAAGCG | |
| | TACGTTCTAGTTGCCAGCC (SEQ ID | |
| | NO: 22) | |
| oSA170 | TCGCCAGGATGCCGGTCAGCATATAT | Forward primer, R9- |
| | CGATACTCAAGGCAGGTCAATTCGCA | Adaptor-BsaXI |
| | CTGTGAGGGTCACATGGGCGTTTGGC | |
| | ACGTACGTTCTAGTTGCCAGCC (SEQ | |
| | ID NO: 23) | |
| oSA171 | ACCGACCTGGGTTCGGCACTGTGGGC | Forward primer, R11- |
| | AGTGTGAGGTATTGGCAGACGCCCAG | Adaptor-BsaXI |
| | TGCCGAACAACACCTGACGTACGTTC | |
| | TAGTTGCCAGCC | |
| | (SEQ ID NO: 24) | |
| oSA172 | GTCTAAGATATGAACTGTTACTCCGCC | Forward primer, |
| | GTTAGGGGCCGCCACCATGGGAT | BsaXI-DNAblock-Rₓ |
| | (SEQ ID NO: 25) | |

### Description of the Plasmids

| Plasmid | |
|---|---|
| name | Description |
| DIAC6964 | Backbone of standard TI front plasmids |
| DIAC6967 | Backbone for standard TI back plasmids |
| D1AD0100 | knob heavy chain single-gene vector |
| D1AD0101 | hole heavy chain single-gene vector |
| D1AD0102 | crossed light chain single-gene vector |
| D1AD0103 | light chain single-gene vector |
| D1AD0274 | Plasmid encoding the template sequence for adaptors. |
| pSA085 | TI basic front vector |
| pSA086 | TI back basic vector |
| pSA087 | TI front vector coding for a TCB, |
| | cloned as described in project 2 |
| pSA089 | TI back vector coding for a TCB, |
| | cloned as described in project 2 |

### DESCRIPTION OF THE CITED DOCUMENTS

[1] Crawford, Y., et al., Biotechnol. Prog. 29 (2013) 1307-1315.
[2] Smith, K., Reprod. Nut. Dev. 41 (2001) 465-485.
[3] Osterlehner, A., et al., Biotechnol. Bioeng. 108 (2011) 2670-2681.
[4] Garrick, D., et al., Nat. Gen. 18 (1998) 56-59.
[5] Dorai, H., et al., Bioprocess Int. 5 (2007) 66.
[6] Nehlsen, K., et al., BMC Biotechnol. 9 (2009) 100.
[7] Barrett, L. W., et al., Cell. Mol. Life Sci. 69 (2012) 3613-3634.
[8] Kozak, M., Cell 44 (1986) 283-292.
[9] Marshall, J. J. and S. E. Halford, The type IIB restriction endonucleases. 2010, Portland Press Limited.
[10] Gibson, D. G., et al., Nat. Meth. 6 (2009) 343.
[11] NEB. NEBuilder® HiFi DNA Assembly Cloning Kit. DNA Modifying Enzymes 2017.
[12] Marshall, J. J. T. and Halford, S. E., Biochem. Soc. Transact. 38 (2010) 410.
[13] Robertson, J. M. and J. Walsh-Weller, An introduction to PCR primer design and optimization of amplification reactions, in Forensic DNA profiling protocols. 1998, Springer. p. 121-154.
[14] Mullis, K., et al. Specific enzymatic amplification of DNA in vitro: the polymerase chain reaction. in Cold Spring Harbor symposia on quantitative biology. 1986. Cold Spring Harbor Laboratory Press.
[15] NEB. Activity of Restriction Enzymes in PCR Buffers.
[16] Roche. High Pure PCR Product Purification Kit. 2017.

### Materials

| | |
|---|---|
| Chemicals | |
| 1 kb Plus DNA Ladder | New English Biolabs ® |
| Agarose MP | Roche Diagnostics GmbH |
| Ampicillin | Sigma-Aldrich ® |
| Bacillol ® AF | BODE Chemie GmbH |
| CutSmart ® Buffer | New English Biolabs ® |
| Ethanol absolute | Merck Chemicals GmbH |
| Ethidium bromide solution 0.07% | AppliChem |
| Gel Loading Dye, Purple (6X) | New English Biolabs ® |
| ImMedia Amp Agar | Invitrogen ™ |
| Isopropanol | Merck Chemicals GmbH |
| NEBuilder ® HiFi DNA Assembly | New English Biolabs ® |
| Master Mix | |
| Nuclease free water | Roche Diagnostics GmbH |
| Premixed TAE Buffer, 10x | Roche Diagnostics GmbH |
| Q5 ® Hot Start High-Fidelity DNA | New English Biolabs ® |
| Polymerase | |
| Shrimp Alkaline Phosphatase (rSAP) | New English Biolabs ® |
| SOC Outgrowth Medium | New English Biolabs ®/ |
| | Invitrogen ™ |
| Competent cell strains | |
| 5-alpha F′Iq Competent E. coli | New English Biolabs ® |
| 10-beta Competent E. coli | New English Biolabs ® |
| (High Efficiency) | |
| ElectroMAX ™ DH5a-E | Invitrogen ™ |
| Competent Cells | |
| One Shot ™ TOP10 Chemically | Invitrogen ™ |
| Competent E. coli | |
| One Shot ™ TOP10 Electrocomp ™ | Invitrogen ™ |
| E. coli | |

| Type II enzymes | Sequence | Overhang |
|---|---|---|
| AfeI | AGC/GCT | Blunt |
| | TCG/CGA | |
| | (SEQ ID NO: 26) | |
| BamHI | G/GATCC | 5′ GATC |
| | CCTAG/G | |
| | (SEQ ID NO: 27) | |
| BsaXI* | NNN/N₉ACN₅CTCCN₇NNN/ | 5′/3′ NNN |
| | /NNNN₉TGN₅GAGGN₇/NNN | |
| | (SEQ ID NO: 28) | |
| BstEII | G/GTNACC | 5′ GTNAC |
| | CCANTG/G | |
| | (SEQ ID NO: 29) | |
| EcoRI | G/AATTC | 5′ AATT |
| | CTTAA/G | |
| | (SEQ ID NO: 30) | |
| HindIII | A/AGCTT | 5′ AGCT |
| | TTCGA/A | |
| | (SEQ ID NO: 31) | |
| | TTAAT/TAA | |
| PacI | AAT/TAATT | 3′ AT |
| | SEQ ID NO: 32) | |
| PspOMI | G/GGCCC | 5′ GGCC |
| | CCCGG/G | |
| | SEQ ID NO: 33) | |
| PvuI | CGAT/CG | 3′ AT |
| | GC/TAGC | |
| | (SEQ ID NO: 34) | |

All Enzymes provided by New English Biolabs® and purchased preferred in high fidelity version; *N=A/T/G/C.

### Kit Name

| | |
|---|---|
| DNA Clean & Concentrator-5 | ZYMO RESEARCH |
| NucleoBond Xtra Maxi EF | Macherey-Nagel |
| QIAprep Spin Miniprep Kit | QIAGEN |
| Quick Ligation ™ Kit | New English Biolabs |
| Zymoclean Gel DNA Recovery Kit | ZYMO RESEARCH |

### Methods

### Primer Design

Primers are single-stranded oligonucleotides of approximately 18-30 nucleotides length. The design is a critical parameter of a successful PCR, thus they are designed according to some guidelines. To amplify a specific DNA sequence without any nonspecific by-products, it is important to create primer pairs, which bind only to one specific site of the template DNA. A primer pair consist of a forward primer (5′-3′ sense strand) and a reverse primer (5′-3′ antisense strand). Once the primer pair anneal to the template DNA, a polymerase elongates the sequence between them and amplifies the DNA strand between the annealing sites. An GC content of 40-60% and a terminal C or G at the 3′-end of the primer sequence increase the specificity and efficiency of priming [13], [14].

By using specific primers, DNA fragments are generated which can be used for subsequent cloning steps. In addition to the binding site, these primers carrying recognition sequences for restriction enzymes and/or recombination sites (R-sites) in their overhangs.

### Restriction Digestion

All restriction enzymes were purchased by NEB and only high fidelity variants were used. Methods were according to the manufacturer's instructions.

### Digestion of PCR Generated Fragments

Designed primers for PCR were used to add recognition sites for restriction enzymes to one end of the fragments. For subsequent ligations, the fragment requires single-stranded overhangs, which were generated via digestion with the respective restriction enzyme. For this, the restriction digestion was prepared directly in the PCR reaction mix. The following table lists a standard protocol for this purpose.

### Exemplary PCR Digestion Conditions of PCR Fragments

| Components | Volume in μL |
|---|---|
| PCR reaction mix | 50 |
| CutSmart ® Buffer | 6 |
| Restriction enzyme | 1 |
| Total volume | 57 |

After an incubation of 15 min to 1 h at 37° C., the samples were purified via AGE or directly via Zymogene DNA Clean & Concentrator Kit.

### Preparative Restriction Digestion and AGE

Digestion reactions were prepared as described in the table below. If several enzymes were used in one digestion, the volume of PCR-grade water was adjusted. The reaction was set on ice and incubated for 15 min to 1 h at a constant temperature of 37° C. in an Eppendorf thermal block.

Exemplary conditions for preparative restriction digestion mix

| Components | Volume in μL |
|---|---|
| DNA (1 μg/μL) | 1 |
| CutSmart ® Buffer (10x) | 5 |
| Each restriction enzyme | 1 |
| ddH2O | to 50 |
| Total volume | 50 |

Afterwards, each sample was mixed with purple loading dye and was transferred onto a 1% agarose electrophoresis gel.

### Analytical Restriction Digestion and AGE

An analytical restriction digestion was used to screen for correct clones after transformation. Therefore, enzymes are selected which cut the DNA into fragments of different sizes. The restriction digestion mix was prepared according to the preparative digestions. After separating the samples with a 1% agarose gel, the lengths of the different fragments were analyzed and compared to the DNA marker and the expected, calculated lengths.

### Polymerase Chain Reaction

PCR is used for enzymatic in vitro amplification of specific DNA fragments from DNA templates. The reaction can be divided into three essential steps, which are repeated several times cyclically. The first phase is “Denaturation” in which the double-stranded DNA is melted by raising the temperature to 95-98° C. The DNA is then available as two single strands. The second step is the “Annealing” phase, which allows the hybridization of two fragment-flanking primers to the 3′-end by lowering the temperature to 55-65° C. Finally, the enzymatic extension of the primers takes place in the “Elongation”, in which the polymerase synthesizes the complementary strand at 72° C. in the 5′-3′ direction. After each cycle, the amplification products are available again as templates. Thus, the increase is exponential [14].

The following table demonstrates the standard programming of the thermal cycler for this project.

### Standard Programming of the Thermal Cycler for PCR.

| Cycle Step | Cycles | T in °C. | t | |
|---|---|---|---|---|
| Initial Denaturation | 1 | 98 | 30 | s |
| Denaturation | 30 | 98 | 10 | s |
| Annealing | | TA | 30 | s |
| Extension | | 72 | 30 | s |
| Final Extension | 1 | 72 | 2 | min |

| | | | |
|---|---|---|---|
| Hold | 1 | 4 | ∞ |

The conditions of PCR vary depending on the length and interactions of primers and template.

PCR with Temperature Gradient

To provide an efficient PCR it's important to know the optional annealing temperature TA of both primers. For this, the sample is tested at different temperatures by setting a temperature gradient in the thermal cycler. The center of the gradient is usually the average of the melting temperatures T_{M} of both primers. In the table below, a standard protocol for temperature gradients in PCR reactions as used in this project is depicted.

Programming of the thermal cycler with temperature gradient:

| Cycle Step | Cycles | T in °C. | t | |
|---|---|---|---|---|
| Initial Denaturation | 1 | 98 | 30 | s |
| Denaturation | 30 | 98 | 10 | s |
| Annealing | | T_{A,1} | 30 | s |
| | | T_{A,2} | | |
| | | T_{A,3} | | |
| | | T_{A,4} | | |
| | | T_{A,5} | | |
| | | T_{A,6} | | |
| | | T_{A,7} | | |
| | | T_{A,8} | | |
| Extension | | 72 | 30 | s/kb |
| Final Extension | 1 | 72 | 2 | min |

| | | | |
|---|---|---|---|
| Hold | 1 | 4 | ∞ |

For both cases of PCR reactions, the elongation time in each cycle was adjusted to the expected longest DNA fragment.

### PCR Purification

The components of a PCR mix are essential for the PCR reaction but may inhibit subsequent modification steps like restriction digestions, ligations, etc. [15]. Therefore, the amplified fragments must be isolated from PCR reaction mix. The purification was performed by the Zymogene DNA Clean & Concentrator Kit. Therefore, the PCR reaction mix was diluted with five volumes of the binding buffer supplied with the Kit. In combination with guanidine salts, the DNA binds selectively to special glass fibers pre-packed in the column. Bound DNA was isolated by washing twice with washing buffer to remove short primers, dNTPs, enzymes, short-failed PCR products, and salts. Finally, the DNA fragments were eluted using a low salt solution [16]. Purity and concentration of the eluted DNA were measured by Nanodrop Spectrophotometer.

### Gel Extraction

The Zymoclean Gel DNA Recovery Kit was used to extract DNA from an agarose gel. Therefore, gel slices including the fragment of interest, were cut out of the agarose gel and placed in a 2 ml Eppendorf cup. The gel slices were dissolved in the dissolving buffer at 50° C. in a thermal block. Afterwards, the solution was pipetted onto the column placed in a tube and was span down 1 min at 13.000 rpm. DNA fragments bind to the silica membrane while other components like salts, enzymes, agarose or other impurities pass the column and were discarded. The columns were washed and centrifuged twice with an ethanol-containing wash buffer. Finally, a variable volume of elution buffer was pipetted directly to the column bed. An incubation of 1 min ensures higher concentrations. After DNA was eluted from the matrix, the final concentration and quality was measured by Nanodrop device.

### Agarose Gel Electrophoresis

In a gel electrophoresis, the DNA is separated by size for visualization or purification. Agarose MP in a final concentration of 0.7-1.2% was added to a 1×TAE buffer and were dissolved by heating in a microwave. Meanwhile the well comb was placed in the gel tray. The addition of ethidium bromide to the agarose mix allows the later visualization of DNA. The mix was transferred in the tray and was sit until it has completely solidified. Afterwards the solid gel was placed in the electrophoresis chamber, which was filled with 1×TAE buffer until the setting line is reached. A DNA ladder was pipetted in the first chamber of the gel by default. The loading dye provided samples were transferred into the different wells and the electrophoresis was run by 110 V for 1.5 h. After completion of the electrophoresis, the gel was removed from the chamber and analyzed in an UV-Imager. The length of the separated DNA was estimated using the DNA marker.

### Assembly Reaction and Transformation for Final Vectors

The assembly reaction was performed by NEBuilder® HiFi DNA Assembly Cloning Kit. Based on the provided equation of the suppliers' protocol (s. Equation), the appropriate DNA amount for each fragment was calculated. A total amount of 0.2 pmol of DNA fragments and a molar ratio of 1:2 vector:insert were chosen for calculation.

$$pmol = \frac{( weight in ng ) \times 1000}{base pairs \times 650 daltons}$$

In following table, an exemplary pipetting scheme is depicted.

### Exemplary Assembly Reaction Mix

| Components | bp | pmol | ng | ng/μL | μL |
|---|---|---|---|---|---|
| Vector | 7587 | 0.067 | 330.0 | 33.7 | 9.79 |
| Insert | 2842 | 2 × 0.067 | 251.0 | 131.8 | 1.90 |
| NEBuilder | | | | | 11.70 |
| HiFi DNA | | | | | |
| Assembly | | | | | |
| Master Mix | | | | | |
| ddH₂O | | | | | 0.00 |
| Total volume | | | | | 23.4 |

After setup the reaction the mix were incubated at 50° C. for 1 h followed by transformation of 2 μL assembled fragments into NEB 10-beta Competent E. coli cells.

### Chemical Transformation

For a chemical transformation, E. coli cells pre-treated with calcium chloride were used. The cells stored in a −80° C. freezer were thawed on ice for around 10 min. Then, 2 ml of ligation or assembly mix were pipetted directly on 50 μL cell mixture and were placed on ice for 30 min. A positive and negative control were prepared simultaneously. After incubation, the cells were heat-shocked at 42° C. for exactly 30 sec and set on ice for 2 min. 250 ml pre-warmed SOC medium was added to the transformation mix and incubated at 37° C. and 300 rpm for 1 h to give the cells time to recover. Meanwhile the selection plates were warmed to 37° C. in an incubator. Finally, 100 to 200 μL of the culture were pipetted on LB-Amp agar plates and spread with plating beads. The plates were incubated overnight at 37° C. Only clones, which incorporated the plasmid carrying the resistance gene against Ampicillin, are able to survive the selection pressure.

### DNA (Mini) Preparation

A preparation of plasmid DNA was used to verify that a clone, which was growing on a selection plate, carries the correct plasmid. Therefore, the purification was performed by QIAprep Spin Miniprep Kit. Single grown colonies were picked with a pipette tip and a culture of 3 ml ampicillin-containing LB-medium was inoculated and incubated overnight at 37° C. The next day, 2 ml of the culture were pipetted in a tube and centrifuged down. The bacterial pellet was resuspended in an alkaline Tris buffer and the suspension was cleared by addition of neutralization buffer and centrifugation. Then, the lysate was transferred to a silica membrane column, which is placed in a tube. In presence of high salt concentrations, the DNA adsorbs to the silica membrane. Impurities were removed by washing with washing buffer. After the flow-through was discarded, the column was centrifuged once empty to remove residues of buffer. Finally, the plasmid DNA was eluted using elution buffer or water and purity and concentration were measured by Nanodrop device.

### DNA (Maxi) Preparation

After obtaining a positive clone, the DNA amount was expanded to receive enough DNA for the transfection mixture. The DNA maxi preparation was performed using the Macherey-Nagel NucleoBond® Xtra Maxi EF Kit. For this, a small amount of culture from mini-prep, which was left in the tube, was transferred to inoculate 300 ml LB-Medium with 0.5 g/L ampicillin. The suspension was incubated overnight at 37° C. and 200 rpm. The next day the LB-Amp medium appeared opaque because of the density of grown bacteria. To separate bacteria from medium the culture was centrifuged in a SLC-3000 rotor at 5600×g at 4° C. for 15 min and the supernatant was discarded. The pellet was resuspended and lysed in an alkaline lysis buffer. The bacterial lysate was cleared via addition of a naturalizing buffer and loaded onto the equilibrated column where the plasmid DNA binds to the anion-exchange resin. The first washing step using a wash buffer is performed with inserted filter to wash out residual lysate from the column. After washing with buffer 1 to eliminate endotoxins and buffer 2 to remove contaminations, the plasmid DNA was eluted in a high-salt elution buffer. With the addition of isopropanol, the plasmid was precipitated and separated as pellet after a centrifugation step for 30 min at 4200×g. The pellet was dissolved in 500 μL of a final buffer. DNA purity and concentration were measured by Nanodrop spectrophotometer.

1. A method for producing an expression vector comprising the following step
incubating
a linear expression vector backbone comprising
at its 3′-end a promoter and a first single stranded enzymatic restriction site of a first restriction enzyme,
at its 5′-end a single stranded recombination site,
a first DNAblock comprising in 5′- to 3′-direction
a first single stranded enzymatic restriction site of a first restriction enzyme,
a nucleic acid encoding a protein of interest,
a first single stranded recombination site,
a first adaptor nucleic acid comprising in 5′- to 3′-direction
a first single stranded recombination site,
a polyA signal sequence,
a promoter,
a second single stranded enzymatic restriction site of a second restriction enzyme,
a second DNAblock comprising in 5′- to 3′-direction
a second single stranded enzymatic restriction site of a second restriction enzyme,
a nucleic acid encoding a second protein of interest,
a second single stranded recombination site,
with a DNA ligase,
whereby the first enzymatic restriction site is different from the second enzymatic restriction site,
whereby the second enzymatic restriction site is of a type IIB restriction enzyme,
whereby the first and the second recombination sites are different,
whereby the second recombination site and the recombination site at the 3′-end of the vector backbone are identical,
wherein each recombination site is a 15-80 bp long nucleic acid sequence unique in the incubated nucleic acids,
wherein the first single stranded enzymatic restriction site of the linear vector backbone is capable of specifically hybridizing with the first single stranded enzymatic restriction site of the first DNAblock, the first single stranded recombination site of the first DNAblock is capable of specifically hybridizing with first single stranded recombination site of the first adaptor nucleic acid, the second single stranded enzymatic restriction site of the first adaptor nucleic acid is capable of specifically hybridizing with the second single stranded enzymatic restriction site of the second DNAblock, and the second single stranded recombination site of the second DNAblock is capable of specifically hybridizing with the single stranded recombination site of the linear vector backbone.

2. The method according to claim 1, wherein the second enzymatic restriction site is a BsaXI restriction site.

3. The method according to claim 1, wherein the protein of interest is an antibody chain.

4. A linear or circular nucleic acid comprising the following elements in 5′- to 3′-direction:
a first promoter nucleic acid sequence,
an enzymatic restriction site of a first restriction enzyme,
a first nucleic acid encoding for a first protein of interest,
a first recombination site,
a first polyadenylation signal sequence,
a second promoter nucleic acid sequence,
an second enzymatic restriction site, which is for a type IIB restriction enzyme,
a second nucleic acid encoding for a second protein of interest,
a second recombination site, and
a second polyadenylation signal sequence,
wherein the first recombination site is different from the second recombination site,
wherein the first enzymatic restriction site is different to the enzymatic restriction site for a type IIB restriction enzyme,
wherein each recombination site is a unique 15-80 bp long nucleic acid sequence.

5. The nucleic acid according to claim 4, wherein the nucleic acid has been obtained with a method according to claim 1.

6. The nucleic acid according to claim 4, wherein the enzymatic restriction site for a type IIB restriction enzyme is a BsaXI restriction site.

7. The nucleic acid according to claim 4, wherein the protein of interest is an antibody chain.

8. A cell comprising the nucleic acid according to claim 4.

9. A method for producing an antibody comprising the following steps:
a) cultivating a mammalian cell comprising the nucleic acid according to claim 7,
b) recovering the antibody from the cell or the cultivation medium,
c) optionally purifying the antibody with one or more chromatography steps, thereby producing the antibody.
